(19) United States
(12) Patent Application Publication
(10) Pub. No.: US 2007/0037828 A1
(43) Pub. Date: Feb. 15, 2007

(54) Pyrazolopyrimidines

(76) Inventors: Olaf Gebauer, Monheim (DE); Ulrich Heinemann, Monheim (DE); Stefan Herrmann, Monheim (DE); Oliver Guth, Monheim (DE); Hans-Ludwig Elbe, Wuppertal (DE); Herbert Gayer, Monheim (DE); Jörg Nico Greul, Monheim (DE); Stefan Hillebrand, Monheim (DE); Ronald Ebbert, Nürnberg (DE); Ulrike Wachendorff-Neumann, Monheim (DE); Peter Dahmen, Monheim (DE); Karl-Heinz Kuck, Monheim (DE)

Correspondence Address: STERNE, KESSLER, GOLDSTEIN & FOX PLLC, 1100 NEW YORK AVENUE, N.W., WASHINGTON, DC, 20005, US

(21) Appl. No.: 10/560,966

(22) PCT Filed: Jun. 18, 2004
(86) PCT No.: PCT/EP04/06609

§ 371 Date: May 31, 2006

(30) Foreign Application Priority Data
Jun. 27, 2003 (DE) 103-28-996.8
Aug. 27, 2003 (DE) 103-39-360.9
Dec. 10, 2003 (DE) 103-57-570.7

(51) Int. Cl. A61K 31/519; C07D 487/04

(52) U.S. Cl. 514/259.3; 544/281

(57) ABSTRACT

New pyrazolopyrimidines of the formula 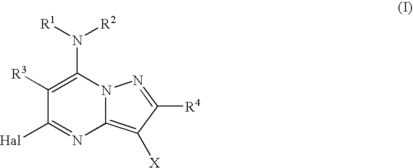 in which
R¹, R², R³, R⁴, X and Hal have the meanings specified in the description, multiple methods for producing these materials and their use for combating undesired micro-organisms.
   New intermediate products of the formulas 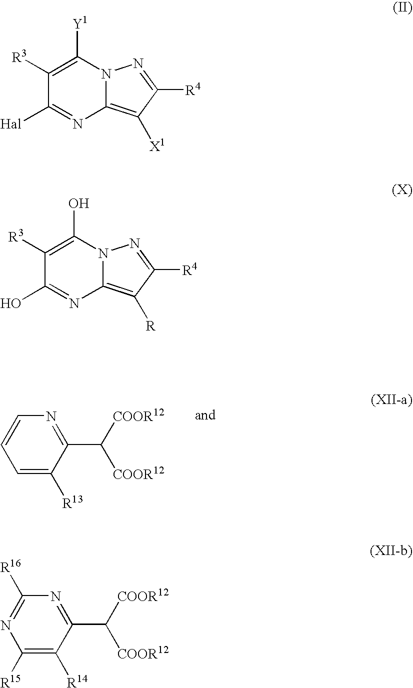 and methods for producing these materials.

The present invention relates to new pyrazolopyrimidines, multiple methods for their production, and their use for combating undesired micro-organisms. In addition, the present invention relates to new intermediate products and methods for their production.

It is already known that specific pyrazolopyrimidines have fungicidal properties (cf. DE-A 3 130 633 or FR-A 2 794 745). The efficiency of these substances is good but in some cases, leaves something to be desired when low quantities are used.

New pyrazolopyrimidines of the formula 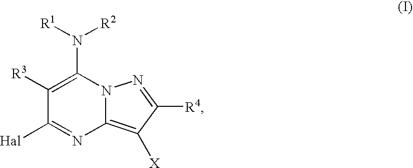
in which
R¹ represents optionally substituted alkyl, optionally substituted alkenyl, optionally substituted alkynyl, optionally substituted cycloalkyl, or optionally substituted heterocyclyl,
R² represents hydrogen or alkyl, or
R¹ and R² together with the nitrogen atom to which they are bound, represent a optionally substituted heterocyclic ring,
R³ represents optionally substituted heterocyclyl,
R⁴ represents hydrogen or alkyl,
Hal represents halogen and
X represents halogen, cyano, nitro, alkyl, optionally substituted alkenyl, optionally substituted alkynyl, hydroxyalkyl, alkoxyalkyl, halogenalkyl, cycloalkyl, formyl, thiocarbamoyl, alkoxycarbonyl, alkylcarbonyl, hydroxyiminoalkyl, alkoximinoalkyl, alkylthio, alkylsulphinyl, alkylsulphonyl or alkylaminocarbonyl have now been found.

The compounds according to the present invention may optionally, depending on the substitution patterns, be provided as mixtures of different possible isomeric forms, particularly stereoisomers, such as E and Z, threo and erythro, and also optical isomers, optionally even in the form of tautomers. If R³ is substituted differently at both atoms which neighbor the connection point, the relevant compounds may be provided in a special form of stereoisomerism, as atropisomers.

Furthermore, it has been found that pyrazolopyrimidines of the formula (I) may be produced by reacting
a) halogen pyrazolopyrimidines of the formula 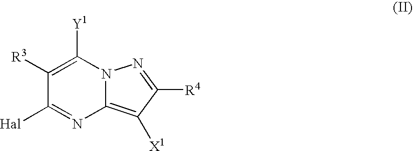
in which
R³, R⁴, and Hal have the meanings specified above,
X¹ represents halogen, cyano, nitro, alkyl, halogenalkyl, cycloalkyl, formyl, thiocarbamoyl, alkoxycarbonyl, alkylcarbonyl, alkylthio, alkylsulphinyl, alkylsulphonyl or alkylaminocarbonyl and
Y¹ represents halogen,
with amines of the formula 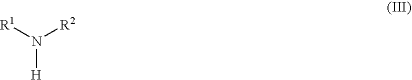
in which
R¹ and R² have the meanings specified above,
optionally in the presence of a diluent, optionally in the presence of a catalyst, and optionally in the presence of an acid acceptor,
   or
   b) pyrazolopyrimidines of the formula 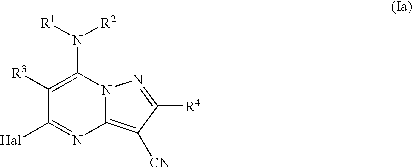
in which
R¹, R², R³, R⁴, and Hal have the meanings specified above,
either
α) are reacted with diisobutyl aluminum hydride in the presence of aqueous ammonium chloride solution and in the presence of an organic diluent,
or
β) are reacted with Grignard compounds of the formula
   R⁵—Mg—X² (IV)
   in which
   R⁵ represents alkyl
   X² represents chloride or bromide,
   in the presence of a diluent and optionally in the presence of a catalyst,
      or
      c) pyrazolopyrimidines of the formula 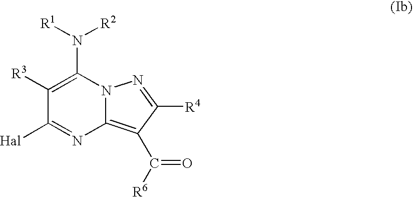
in which
R¹, R², R³, R⁴, and Hal have the meanings specified above and
R⁶ represents hydrogen or alkyl,
either
α) are reacted with amino compounds of the formula
   H₂N—OR⁷ (V)
   in which
   R⁷ represents hydrogen or alkyl,
   in the presence of a diluent and optionally in the presence of a catalyst, the amino compounds of the formula (V) also being able to be used in the form of their acid addition salts,
or
β) are reacted with triphenylphosphonium salts of the formula 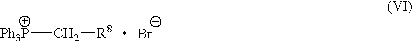
   in which
   Ph represents phenyl and
   R⁸ represents hydrogen or optionally substituted alkyl,
   in the presence of a base and in the presence of a diluent,
   or
γ) are reacted with diisobutyl aluminum hydride in the presence of aqueous ammonium chloride solution and in the presence of an organic diluent,
   or are reacted with sodium borohydride in the presence of a diluent,
   and optionally the resulting pyrazolopyrimidines of the formula 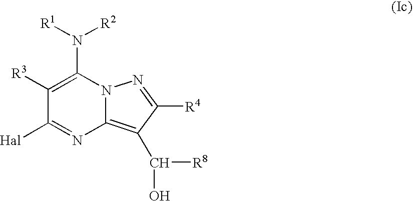
   in which
   R¹, R², R³, R⁴, R⁸, and Hal have the meanings specified above,
   are reacted with alkylation agents of the formula
      R⁹—X³ (VII)
   in which
   R⁹ represents alkyl

X³ represents chloride, bromide, iodide or the residue R⁹⁰—SO₂—O—,
optionally in the presence of a base and in the presence of a diluent,
or
   d) pyrazolopyrimidines of the formula 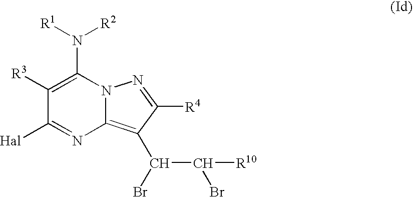
in which
R¹, R², R³, R⁴ and Hal have the meanings specified above,
R¹⁰ represents hydrogen or optionally substituted alkyl,
are reacted with strong bases in the presence of a diluent,
or
   e) pyrazolopyrimidines of the formula 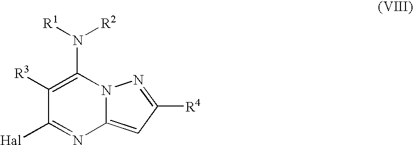
in which
R¹, R², R³, R⁴ and Hal have the meanings specified above,
are reacted with acyl derivates of the formula 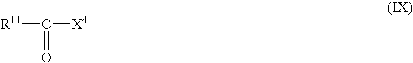
in which
R¹¹ represents alkyl and
X⁴ represents chloride or a residue of the formula 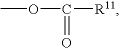
in the presence of a catalyst and in the presence of a diluent.

Finally, it has been found that pyrazolopyrimidines of the formula (I) are very well suitable for combating undesired micro-organisms. Above all, they display a strong fungicidal activity and may be used both in plant protection and also in material protection.

Surprisingly, the pyrazolopyrimidines of the formula (I) have a significantly better microbicidal activity than the most constitutionally similar previously known materials of identical direction of activity.

The compounds of the formula (I) according to the present invention may optionally be provided as mixtures of different possible isomeric forms, particularly stereoisomers, such as E and Z, threo and erythro, and also optical isomers, such as R and S isomers or atropisomers, optionally even tautomers.

Both the pure stereoisomers and any arbitrary mixtures of these isomers are the object of the present invention, even if only compounds of the formula (I) are generally discussed here.

Depending on the type of the substituents defined above, the compounds of the formula (I) have acid or basic properties and may form salts. If the compounds of the formula (I) carry hydroxy, carboxy, or other groups which induce acid properties, these compounds may be reacted with bases to produce salts. Suitable bases are, for example, hydroxides, carbonates, hydrogen carbonates of the alkaline and alkaline earth metals, particularly those of sodium, potassium, magnesium, and calcium, as well as ammonia, primary, secondary, and tertiary amines having (C₁-C₄) alkyl residues as well as mono-, di-, and trialkanolamines of (C₁-C₄) alkanols. If the compounds of the formula (I) have amino, alkylamino, or other groups inducing basic properties, these compounds may be reacted with acids to produce salts. Suitable acids are, for example, mineral acids, like hydrochloric acid, sulphuric acid, and phosphoric acid, organic acids such as acetic acid or oxalic acid, and acid salts, such as NaHSO₄ and KHSO₄. The salts which may thus be obtained also have fungicidal and microbicidal properties.

The object of the present invention is also the salt-like derivatives produced from compounds of the formula (I) through reaction with the basic and/or acidic compounds as well as the N oxides producible according to typical oxygenation methods.

In the present case, heterocyclyl represents saturated or unsaturated, aromatic or non-aromatic cyclic compounds having 3 to 8 ring members, in which at least one ring member represents a heteroatom, i.e., an atom different from carbon. If the ring contains multiple heteroatoms, these may be identical or different. Heteroatoms are preferably oxygen, nitrogen, or sulphur. If the ring contains multiple oxygen atoms, these are not directly neighboring. The cyclic compounds optionally jointly form a polycyclic ring system with further carbocyclic or heterocyclic, fused or bridged rings. Monocyclic or bicyclic ring systems, particularly monocyclic or bicyclic aromatic ring systems are preferred.

The pyrazolopyrimidines according to the present invention are generally defined by the formula (I). Those materials of the formula (I), in which
R¹ represents alkyl having 1 to 6 carbon atoms, which may be substituted one to five times, identically or differently, by halogen, cyano, hydroxy, alkoxy having 1 to 4 carbon atoms and/or cycloalkyl having 3 to 6 carbon atoms, or
R¹ represents alkenyl having 2 to 6 carbon atoms, which may be substituted one to three times, identically or differently by halogen, cyano, hydroxy, alkoxy having 1 to 4 carbon atoms and/or cycloalkyl having 3 to 6 carbon atoms, or
R¹ represents alkynyl having 2 to 6 carbon atoms, which may be substituted one to three times, identically or differently by halogen, cyano, alkoxy having 1 to 4 carbon atoms and/or cycloalkyl having 3 to 6 carbon atoms, or
R¹ represents cycloalkyl having 3 to 6 carbon atoms, which may be substituted one to three times, identically or differently by halogen and/or alkyl having 1 to 4 carbon atoms, or
R¹ represents saturated or unsaturated heterocyclyl having 5 or 6 ring members and 1 to 3 heteroatoms, such as nitrogen, oxygen, and/or sulphur, the heterocyclyl able to be substituted once or twice by halogen, alkyl having 1 to 4 carbon atoms, cyano, nitro and/or cycloalkyl having 3 to 6 carbon atoms,
R² represents hydrogen or alkyl having 1 to 4 carbon atoms, or
R¹ and R² together with the nitrogen atom to which they are bound, represent a saturated or unsaturated heterocyclic ring having 3 to 6 ring elements, the heterocyclic compound able to contain a further nitrogen, oxygen, or sulphur atom as a ring element and the heterocyclic compound able to be substituted up to three times by fluoride, chloride, bromide, nitro, alkyl having 1 to 4 carbon atoms and/or halogenalkyl having 1 to 4 carbon atoms and 1 to 9 fluorine and/or chlorine atoms,
R³ represents saturated or unsaturated heterocyclyl having 5 or 6 ring members and 1 to 4 heteroatoms, such as oxygen, nitrogen and/or sulphur, the heterocyclyl being able to be substituted one to four times, identically or differently by
fluoride, chloride, bromide, cyano, nitro, alkyl, alkoxy, hydroximinoalkyl or alkoximinoalkyl each having 1 to 3 carbon atoms in each alkyl part,
halogenalkyl or halogenalkoxy each having 1 to 3 carbon atoms and 1 to 7 halogen atoms,
R⁴ represents hydrogen or alkyl having 1 to 4 carbon atoms
Hal represents fluoride, chloride, or bromide and
X represents cyano, fluoride, chloride, bromide, iodide, nitro, formyl, halogenalkyl having 1 to 6 carbon atoms and 1 to 9 fluoride, chloride and/or bromide atoms, alkyl having 1 to 4 carbon atoms, alkenyl having 2 to 6 carbon atoms, alkenyl, substituted by carboxyl, methoxycarbonyl, or ethoxycarbonyl, having 2 to 5 carbon atoms in the alkenyl part, alkynyl having 2 to 6 carbon atoms, alkenyl, substituted by carboxyl, methoxycarbonyl, or ethoxycarbonyl, having 2 to 5 carbon atoms in the alkynyl part, hydroxyalkyl having 1 to 4 carbon atoms, alkoxyalkyl having 1 to 4 carbon atoms in the alkoxy part and 1 to 4 carbon atoms in the alkyl part, cycloalkyl having 3 to 6 carbon atoms, thiocarbamoyl, alkoxycarbonyl having 1 to 4 carbon atoms in the alkoxy part, alkylcarbonyl having 1 to 4 carbon atoms in the alkyl part, hydroximinoalkyl having 1 to 4 carbon atoms in the alkyl part, alkoximinoalkyl having 1 to 4 carbon atoms in the alkoxy part and 1 to 4 carbon atoms in the alkyl part, alkylthio having 1 to 4 carbon atoms, alkylsulphinyl having 1 to 4 carbon atoms, alkylsulphonyl having 1 to 4 carbon atoms or alkylaminocarbonyl having 1 to 4 carbon atoms in the alkyl part, are especially preferred.

Those pyrazolopyrimidines of the formula (I), in which
R¹ represents a residue of the formula 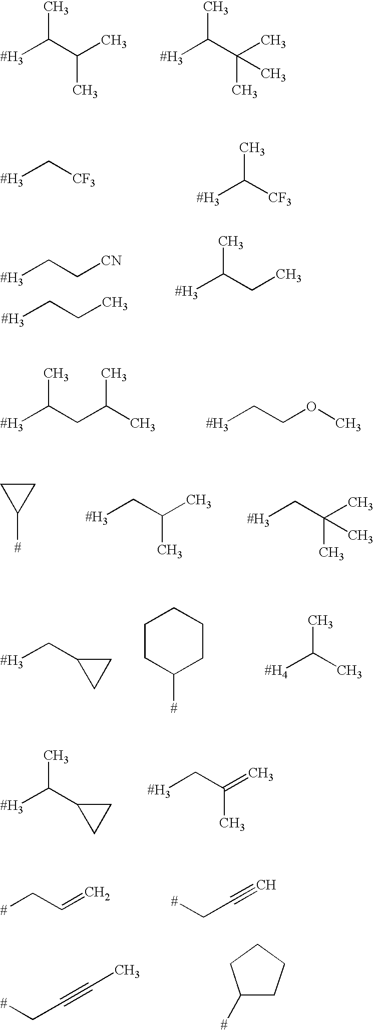
R² represents hydrogen, methyl, ethyl or propyl, or
R¹ and R² together with the nitrogen atom to which they are bound, represent pyrrolidinyl, piperidinyl, morpholinyl, thiomorpholinyl, piperazinyl, 3,6-dihydro-1(2H)-piperidinyl or tetrahydro-1(2H)-pyridazinyl, these residues being able to be substituted by 1 to 3 fluoride atoms, 1 to 3 methyl groups and/or trifluoromethyl,
or
R¹ and R² together with the nitrogen atom to which they are bound, represent a residue of the formula 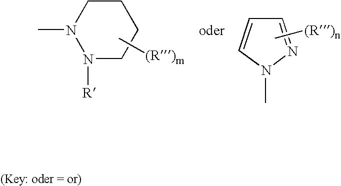
   R′ represents hydrogen or methyl,
   R″ represents methyl, ethyl, fluorine, chlorine or trifluoromethyl,
   m represents the numbers 0, 1, 2 or 3, R″ representing identical or different residues if m represents 2 or 3,
   R′″ represents methyl, ethyl, fluorine, chlorine or trifluoromethyl and
   n represents the numbers 0, 1, 2 or 3, R′″ representing identical or different residues if n represents 2 or 3,
R³ represents pyridyl, which is linked in the second or fourth position and may be substituted one to four times, identically or differently, by fluoride, chloride, bromide, cyano, nitro, methyl, ethyl, methoxy, methylthio, hydroximinomethyl, hydroximinoethyl, methoximinomethyl, methoximinoethyl and/or trifluoromethyl, or
R³ represents pyrimidyl, which is linked in the second or fourth position and may be substituted one to three times, identically or differently, by fluoride, chloride, bromide, cyano, nitro, methyl, ethyl, methoxy, methylthio, hydroximinomethyl, hydroximinoethyl, methoximinomethyl, methoximinoethyl and/or trifluoromethyl, or
R³ represents thienyl, which is linked in the second or third position and may be substituted one to three times, identically or differently, by fluoride, chloride, bromide, cyano, nitro, methyl, ethyl, methoxy, methylthio, hydroximinomethyl, hydroximinoethyl, methoximinomethyl, methoximinoethyl and/or trifluoromethyl, or
R³ represents thiazolyl, which is linked in the second, fourth, or fifth position and may be substituted once or twice, identically or differently, by fluoride, chloride, bromide, cyano, nitro, methyl, ethyl, methoxy, methylthio, hydroximinomethyl, hydroximinoethyl, methoximinomethyl, methoximinoethyl and/or trifluoromethyl,
R⁴ represents hydrogen, methyl, ethyl, propyl or isopropyl
Hal represents fluoride or chloride and
X represents cyano, fluoride, chloride, bromide, iodide, nitro, formyl, trifluoromethyl, difluoromethyl, methyl, ethyl, cyclopropyl, thiocarbamoyl, methoxycarbonyl, methylcarbonyl, ethylcarbonyl, hydroximinomethyl, methoximinomethyl, methylthio, methylsulphinyl methylsulphonyl, methylaminocarbonyl, ethenyl, propenyl, hydroxymethyl, hydroxyeth-1-yl, methoxymethyl, ethoxymethyl or 1-methoxy-ethyl, or
X represents a residue of the formula

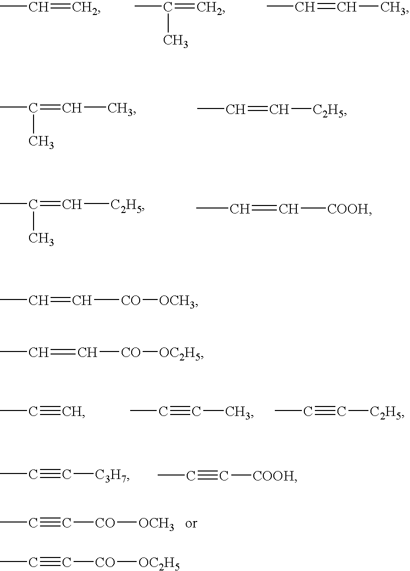

The above-mentioned residue definitions may be combined arbitrarily with one another. In addition, individual definitions may be dispensed with.

If one uses 3-cyano-5,7-dichloro-6-(3-trifluoromethyl-pyridin-2-yl)-pyrazolo[1,5-a]pyrimidine and 2,2,2-trifluoroisopropylamine as starting materials, the course of the method (a) according to the present invention may be illustrated by the following formula scheme.

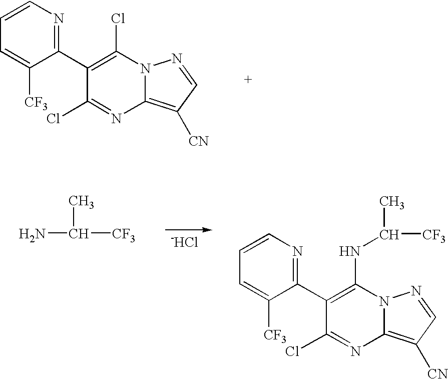

If one uses 3-cyano-5-chloro-6-(3-trifluoromethyl-pyridin-2-yl)-7-(2,2,2-trifluoroisopropylamino)-pyrazolo[1,5-a]pyrimidine as a starting material and diisobutyl aluminum hydride as a reaction component, the course of the method (b, variation α) according to the present invention may be illustrated by the following formula scheme.

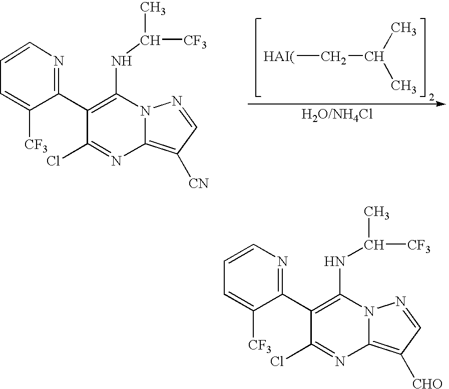

If one uses 3-cyano-5-chloro-6-(3-trifluoromethyl-pyridin-2-yl)-7-(2,2,2-trifluoroisopropylamino)-pyrazolo[1,5-a]pyrimidine as a starting material and methyl magnesium bromide as a reaction component, the course of the method (b, variation β) according to the present invention may be illustrated by the following formula scheme.

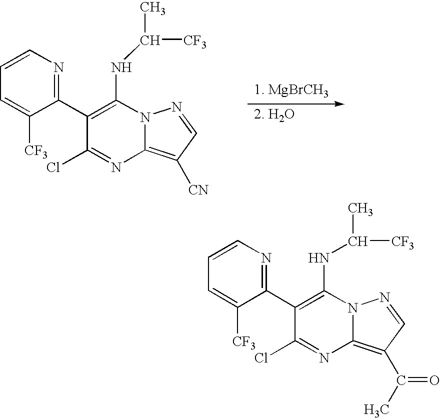

If one uses 3-formyl-5-chloro-6-(3-trifluoromethyl-pyridin-2-yl)-7-(2,2,2-trifluoroisopropylamino)-pyrazolo-[1,5a]pyrimidine and methoxyamine hydrochloride as the starting materials, the course of the method (c, variation α) according to the present invention may be illustrated by the following formula scheme.

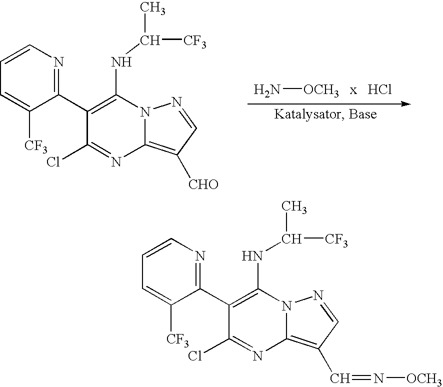

If one uses 3-methylcarbonyl-5-chloro-6-(3-trifluoromethyl-pyridin-2-yl)-7-(2,2,2-trifluoroisopropylamino)-pyrazolo-[1,5a]pyrimidine as the starting material and triphenyl methyl phosphonium bromide as a reaction component, the course of the method (c, variation β) according to the present invention may be illustrated by the following scheme.

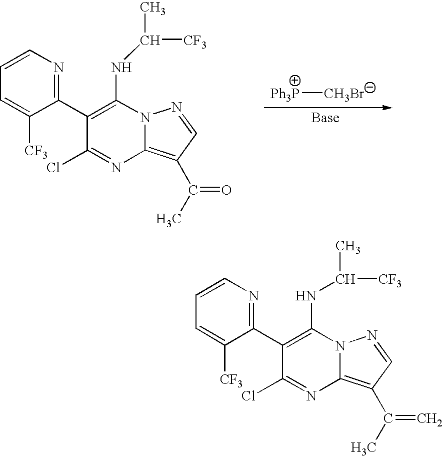

If one uses 3-methylcarbonyl-5-chloro-6-(3-trifluoromethyl-pyridin-2-yl)-7-(2,2,2-trifluoroisopropylamino)-pyrazolo-[1,5a]pyrimidine as a starting material, diisobutyl aluminum hydride as a reaction component in the first step and methyl iodide as a reaction component in the second step, the course of the method (c, variation γ) according to the present invention may be illustrated by the following formula scheme.

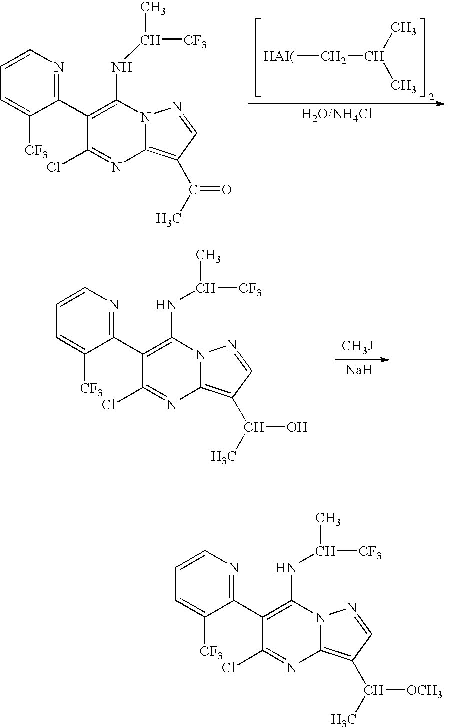

If one uses 3-(1,2-dibromopropyl)-5-chloro-6-(5-chloro-pyrimidin-4-yl)-7-(4-methyl-piperidino)-pyrazolo-[1,5a]pyrimidine as a starting material and potassium tert.-butylate as a reaction component, the course of the method (d) according to the present invention may be illustrated by the following formula scheme.

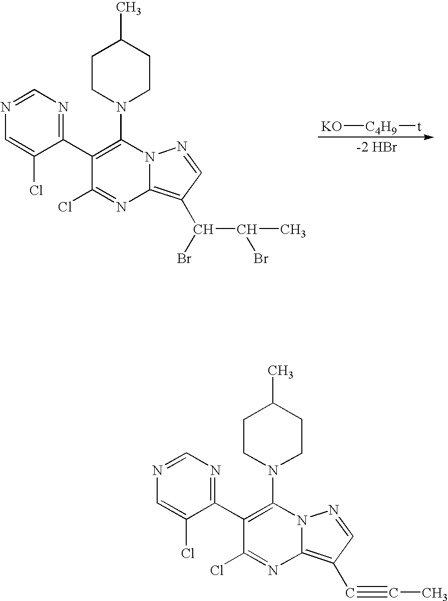

If one uses 5-chloro-6-(5-chloro-pyrimidin-4-yl)-7-(4-methyl-piperidino)-pyrazolo-[1,5a]pyrimidine as a starting material, acetylchloride as a reaction component and aluminum trichloride as a catalyst, the course of the method (e) according to the present invention may be illustrated by the following formula scheme.

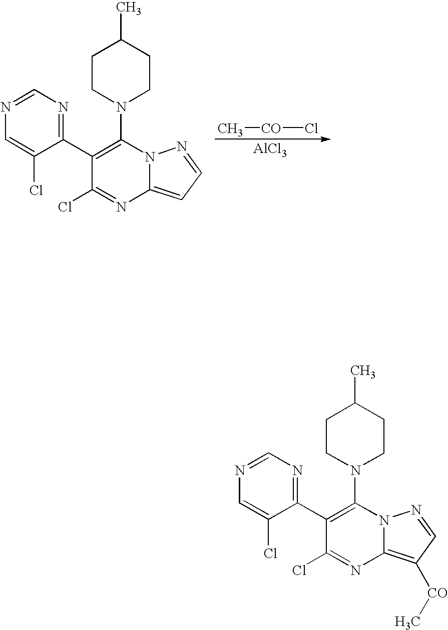

The halogen pyrazolopyrimidines necessary as starting materials for performing the method (a) according to the present invention are generally defined by the formula (II). In this formula (II), R³, R⁴ and Hal preferably have those meanings which were already cited as preferred for these residues in connection with the description of the compounds according to the present invention of the formula (I).
Y¹ preferably represents fluoride, chloride, or bromide, especially preferably fluoride or chloride.
X¹ preferably represents cyano, fluoride, chloride, bromide, iodide, nitro, halogenalkyl having 1 to 6 carbon atoms and 1 to 9 fluoride, chloride, and/or bromide atoms, alkyl having 1 to 4 carbon atoms, formyl, cycloalkyl having 3 to 6 carbon atoms, thiocarbamoyl, alkoxycarbonyl having 1 to 4 carbon atoms in the alkoxy part, alkylcarbonyl having 1 to 4 carbon atoms in the alkyl part, alkylthio having 1 to 4 carbon atoms, alkylsulphinyl having 1 to 4 carbon atoms, alkylsulphonyl having 1 to 4 carbon atoms or alkylaminocarbonyl having 1 to 4 carbon atoms in the alkyl part.
X¹ especially preferably represents cyano, fluoride, chloride, bromide, iodide, nitro, trifluoromethyl, difluoromethyl, methyl, ethyl, formyl, cyclopropyl, thiocarbamoyl, methoxycarbonyl, methylcarbonyl, methylthio, ethylcarbonyl, methylsulphinyl, methylsulphonyl, methylaminocarbonyl, 1,2-dibromopropyl, or 1,2-dibromobutyl.

The halogen pyrazolopyrimidines of the formula (II) are new. These materials are also suitable for combating undesired micro-organisms.

The halogen pyrazolopyrimidines of the formula (II) may be produced,

by reacting

f) hydroxy pyrazolopyrimidines of the formula 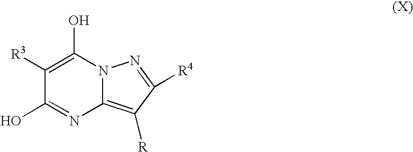
in which
R³ and R⁴ have the meanings specified above, and
R represents halogen, cyano, nitro, alkyl, halogenalkyl, cycloalkyl, thiocarbamoyl, alkoxycarbonyl, alkylthio, alkylsulphinyl, alkylsulphonyl or alkylaminocarbonyl,
with halogenation agents, optionally in the presence of a diluent,
or
   g) by reacting hydroxy pyrazolopyrimidines of the formula 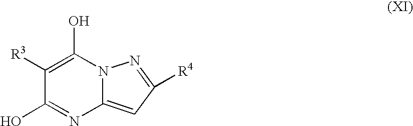
in which
R³ and R⁴ have the meanings specified above,
with phosphorus oxychloride in the presence of dimethylformamide and optionally reacting further while adding phosphorus pentachloride.

If one uses 3-cyano-6-(3-trifluoromethyl-pyridin-2-yl)-pyrazolo[1,5-a]pyrimidin-5,7-diol as a starting material and phosphorus oxychloride mixed with phosphorus pentachloride as a halogenation agent, the course of the method (f) according to the present invention may be illustrated by the following formula scheme.

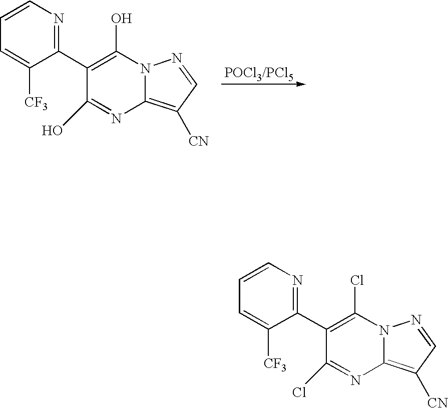

The hydroxy pyrazolopyrimidines necessary as starting materials for performing the method (f) according to the present invention are generally defined by the formula (X). In this formula, R³ and R⁴ preferably have those meanings which were already cited as preferred for these residues in connection with the description of the compounds according to the present invention of the formulas (I). R preferably represents cyano, fluoride, chloride, bromide, iodide, nitro, alkyl having 1 to 4 carbon atoms, halogenalkyl having 1 to 4 carbon atoms and 1 to 9 fluoride, chloride, and/or bromide atoms, cycloalkyl having 3 to 6 carbon atoms, thiocarbamoyl, alkoxycarbonyl having 1 to 4 carbon atoms in the alkoxy part, alkylthio having 1 to 4 carbon atoms, alkylsulphinyl having 1 to 4 carbon atoms, alkylsulphonyl having 1 to 4 carbon atoms or alkylaminocarbonyl having 1 to 4 carbon atoms in the alkyl part.
R especially preferably represents cyano, fluoride, chloride, bromide, iodide, nitro, trifluoromethyl, difluoromethyl, chloromethyl, methyl, ethyl, cyclopropyl, thiocarbamoyl, methoxycarbonyl, methylcarbonyl, methylthio, ethylcarbonyl, methylsulphinyl, methylsulphonyl or methylaminocarbonyl.

The hydroxy pyrazolopyrimidines of the formula (X) are also previously unknown. They may be produced by reacting
(h) heterocyclyl malonic esters of the formula 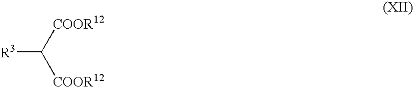
in which
R³ has the meanings specified above and
R¹² represents alkyl having 1 to 4 carbon atoms,
   with aminopyrazolen of the formula 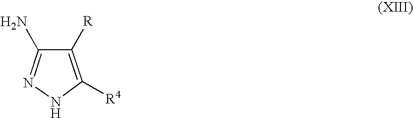
   in which
R⁴ and R have the meanings specified above,
   optionally in the presence of a diluent and optionally in the presence of an acid binder.

If one uses 2-(3-trifluoromethyl-pyridin-2-yl)malonic dimethylester and 3-amino-4-cyano-pyrazole as the starting materials, the course of the method (h) according to the present invention may be illustrated by the following formula scheme.

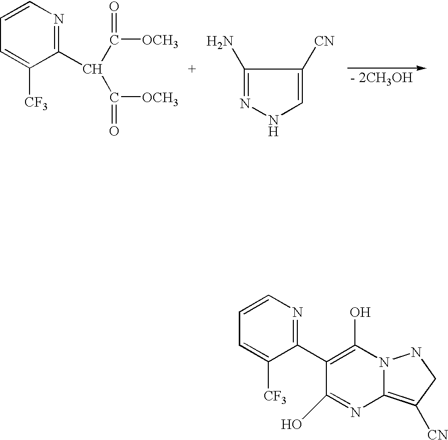

The heterocyclyl malonic esters necessary as starting materials for performing the method (h) according to the present invention are generally defined by the formula (XII). In this formula, R³ preferably has those meanings which were already cited as preferred for this residue in connection with the description of the materials according to the present invention of the formula (I). R¹² preferably represents methyl or ethyl.

The heterocyclyl malonic esters of the formula (XII) are partially known (cf. DE 38 20 538-A, WO 01-11 965 and WO 99-32 464).

Pyridyl malonic esters of the formula 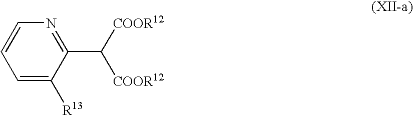
in which
R¹² has the meaning specified above and
R¹³ represents halogen or halogenalkyl are new.

Pyrimidyl malonic esters of the formula 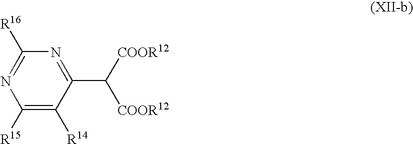
in which
R¹² has the meaning specified above,
R¹⁴ represents halogen or halogenalkyl, and
R¹⁵ and R¹⁶ independently of one another, represent hydrogen, fluoride, chloride, bromide, methyl, ethyl or methoxy, are also new.

The pyridyl malonic esters of the formula (XII-a) may be produced by
(i) reacting halopyridines of the formula 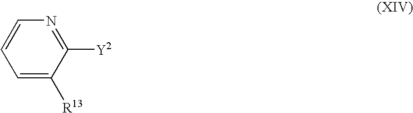
in which
R¹³ has the meaning specified above and
Y² represents halogen,
   with malonic esters of the formula 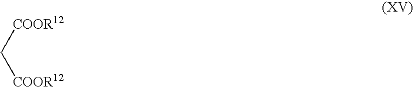
   in which
R¹² has the meaning specified above,
   optionally in the presence of a diluent, optionally in the presence of a copper salt, and optionally in the presence of an acid acceptor.

If one uses 2-chloro-3-trifluoromethylpyridine and malonic acid dimethylester as the starting materials, the course of the method (i) according to the present invention may be illustrated by the following formula scheme.

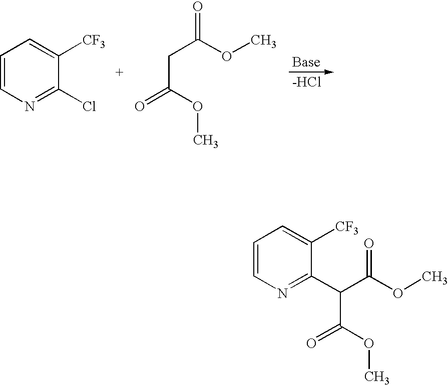

The halopyridines necessary as starting materials for performing the method (i) according to the present invention are generally defined by the formula (XIV). In this formula, R¹³ preferably represents fluoride, chloride or trifluoromethyl. Y² preferably represents chloride or bromide.

The halopyridines of the formula (XIV) are known synthetic chemicals.

The malonic acid esters of the formula (XV), also necessary as starting materials for performing the method (i) according to the present invention, are also known synthetic chemicals.

The pyrimidyl malonic esters of the formula (XII-b) may be produced by reacting
(j) halopyrimidines of the formula 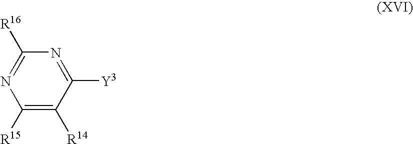
in which
R¹⁴, R¹⁵ and R¹⁶ have the meanings specified above and
Y³ represents halogen,
   with malonic esters of the formula 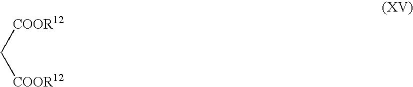
   in which
R¹² has the meaning specified above,
   optionally in the presence of a diluent, optionally in the presence of a copper salt, and optionally in the presence of an acid acceptor.

If one uses 4,5-dichloropyrimidine and malonic dimethylester as the starting materials, the course of the method (1) according to the present invention may be illustrated by the following formula scheme.

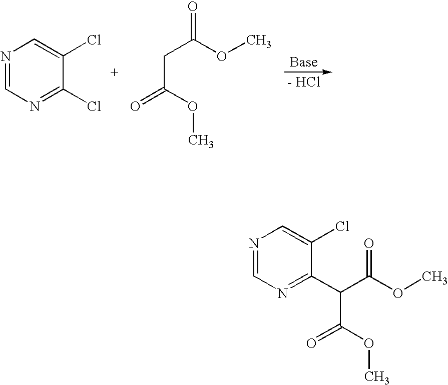

The halopyrimidines necessary as starting materials for performing the method (O) according to the present invention are generally defined by the formula (XVI). In this formula, R¹⁴ preferably represents fluoride, chloride or trifluoromethyl. R¹⁵ and R¹⁶ also, independently of one another, preferably represent hydrogen, fluoride, chloride, bromide, methyl, ethyl or methoxy. Y³ preferably represents chloride or bromide.

The halopyrimidines of the formula (XVI) are known or may be produced according to known methods (cf. J. Chem. Soc. 1955, 3478-3481).

The aminopyrazoles necessary as reaction components for performing the method (h) according to the present invention are generally defined by the formula (XIII). In this formula, R⁴ preferably has those meanings which were already cited as preferred for this residue in connection with the description of the materials of the formula (I) according to the present invention. R preferably has those meanings which were already cited as preferred for this residue in connection with the description of the hydroxy pyrazolopyrimidines of the formula (X).

The aminopyrazoles of the formula (XIII) are known or may be produced according to known methods

All components typical for replacing hydroxy groups with halogen come into consideration as the halogenation agents when performing the method (f) according to the present invention. Phosphorus trichloride, phosphorus tribromide, phosphorus pentachloride, phosphorus oxychloride, thionyl chloride, thionyl bromide or their mixtures are preferably usable. The corresponding fluoride compounds of the formula (II) may be produced from the chloride or bromide compounds through reaction with potassium fluoride.

The halogenation agents cited are known.

The method (g) is suitable for producing halogen pyrazolopyrimidines of the formula 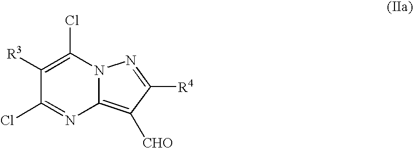
in which
R³ and R⁴ have the meanings specified above.

The hydroxy pyrazolopyrimidines necessary as starting materials for performing the method (g) are generally defined by the formula (XI). In this formula, R³ and R⁴ preferably have those meanings which were already specified as preferred for these residues in connection with the description of the materials of the formula (I) according to the present invention.

The hydroxy pyrazolopyrimidines of the formula (XI) may be produced according to the method (h) by using aminopyrazoles of the formula (XII), in which R represents hydrogen.

The method (g) is performed under the conditions of the Vilsmeier formulation with the aid of phosphorus oxychloride in the presence of dimethylformamide. Phosphorus pentachloride may also be added as a chlorination agent in this case.

The reaction temperatures may be varied in a large range when performing the method (g). In general, one operates at between −10° C. and +150° C., preferably between 0° C. and 120° C.

When performing the method (g), one generally uses 2 to 5 mol of dimethyl formamide, 5 to 15 mol phosphorus oxychloride and optionally 0 two 2 mol phosphorus pentachloride for 1 mol of hydroxy pyrazolopyrimidines of the formula (XI). The workup is performed according to typical methods.

The amines also necessary as starting materials for performing the method (a) according to the present invention are generally defined by the formula (III). In this formula, R¹ and R² preferably have those meanings which were already specified as preferred for R¹ and R² in connection with the description of the compounds of the formula (I) according to the present invention.

The amines of the formula (III) are known or may be produced according to known methods.

All typical inert organic solvents come into consideration as diluents when performing the method (a) according to the present invention. Halogenated hydrocarbons are preferably usable, such as chlorobenzene, dichlorobenzene, dichloromethane, chloroform, tetrachloromethane, dichloroethane or trichloroethane; ethers, such as diethylether, diisopropylether, methyl-t-butylether, methyl-t-amylether, dioxane, tetrahydrofuran, 1,2-dimethoxyethane, 1,2-diethoxyethane or anisol; nitriles, such as acetonitrile, propionitrile, n- or i-butyronitrile or benzonitrile; amides, such as N,N-dimethylformamide, N,N-dimethylacetamide, N-methylformanilide, N-methylpyrrolidone or hexamethyl phosphoric triamide; esters such as acetic methyl ester or acetic ethyl ester; sulphoxides, such as dimethylsulphoxide; sulphones, such as sulpholan.

All inorganic or organic bases typical for reactions in this type come into consideration as acid receptors when performing the method (a) according to the present invention. Alkaline earth metal or alkali metal hydrides, hydroxides, amides, alcoholates, acetates, carbonates or hydrogen carbonates, such as sodium hydride, sodium amide, lithium diisopropylamide, sodium methylate, sodium ethylate, potassium tert.-butylate, sodium hydroxide, potassium hydroxide, sodium acetate, potassium acetate, calcium acetate, sodium carbonate, potassium carbonate, potassium hydrogen carbonate and sodium hydrogen carbonate, and additionally ammonium compounds wie ammonium hydroxide, ammonium acetate and ammonium carbonate, as well as tertiary amines, such as trimethylamine, triethylamine, tributylamine, N,N-dimethylaniline, N,N-dimethyl-benzylamine, pyridine, N-methylpiperidine, N-methylmorpholine, N,N-dimethylaminopyridine, diazabicyclooctane (DABCO), diazabicyclononene (DBN) or diazabicycloundecene (DBU) are preferably usable.

All typical reaction accelerators for reactions of this type come into consideration as catalysts when performing the method (a) according to the present invention. Fluorides such as sodium fluoride, potassium fluoride, or ammonium fluoride are preferably usable.

The reaction temperatures may be varied in a wide range when performing the method (a) according to the present invention. In general, one operates at temperatures between 0° C. and 150° C., preferably at temperatures between 0° C. and 80° C.

When performing the method (a) according to the present invention, generally 0.5 to 10 mol, preferably 0.8 to 2 mol of amine of the formula (III) is used for 1 mol of dihalogen pyrazolopyrimidine of the formula (II). The workup is performed according to typical methods.

The pyrazolopyrimidines necessary as starting materials when performing the second step of the method (b) according to the present invention are generally defined by the formula (Ia). In this formula, R¹, R², R³, R⁴ and Hal preferably have those meanings which were already cited in connection with the description of the materials according to the present invention of the formula

The pyrazolopyrimidines of the formula (Ia) are materials according to the present invention which may be produced according to the method (a) according to the present invention.

All typical inert, organic solvents come into consideration as the diluent when performing the method (b, variation α) according to the present invention. Aliphatic or aromatic, optionally halogenated hydrocarbons, such as toluene, dichloromethane, chloroform or carbon tetrachloride are preferably usable.

The reaction temperatures may be varied within a specific range when performing the method (b, variation α) according to the present invention. In general, one operates at temperatures between −80° C. and +20° C., preferably between −60° C. and +10° C.

When performing the method (b, variation α) according to the present invention, generally an equivalent quantity or even an excess, preferably 1.1 to 1.2 mol diisobutyl aluminum hydride is used for 1 mol of pyrazolopyrimidine of the formula (Ia) and subsequently an excess of aqueous ammonium chloride solution is added. The workup is performed according to typical methods. Generally, the reaction mixture is acidified, the organic phase is separated, the aqueous phase is extracted using an organic solvent slightly miscible with water, and the combined organic phases are washed, dried, and concentrated under reduced pressure.

The Grignard compounds necessary as reaction components when performing the method (b, variation β) according to the present invention are generally defined by the formula (IV). In this formula, R⁵ preferably represents alkyl having 1 to 4 carbon atoms, especially preferably methyl or ethyl. X² preferably represents bromide.

All reaction accelerators typical for Grignard reactions of this type come into consideration as catalysts when performing the method (b, variation β) according to the present invention. Examples are potassium iodide and iodide.

All inert organic solvents typical for reactions of this type come into consideration as the diluent when performing the method (b, variation β) according to the present invention. Ethers, such as diethylether, dioxane or tetrahydrofuran, are preferably usable, as well as aromatic hydrocarbons, such as toluene, and also mixtures of ethers and aromatic hydrocarbons, such as toluene/tetrahydrofuran.

The reaction temperatures may be varied in a specific range when performing the method (b, variation β) according to the present invention. Generally, one operates at temperatures between −20° C. and +100° C., preferably between 0° C. and 80° C.

When performing the method (b, variation β) according to the present invention, 2 to 3 mol of Grignard compound of the formula (IV) is generally used for 1 mol of pyrazolopyrimidine of the formula (Ia). An aqueous workup is subsequently performed according to typical methods.

The pyrazolopyrimidines necessary as starting materials for performing the method (c) according to the present invention are generally defined by the formula (Ib). In this formula, R¹, R², R³, R⁴ and Hal preferably have those meanings which were already cited as preferred for these residues in connection with the description of the materials according to the present invention of the formula (I). R⁶ preferably represents hydrogen or alkyl having 1 to 4 carbon atoms, especially preferably hydrogen, methyl or ethyl.

The pyrazolopyrimidines of the formula (Ib) are materials according to the present invention which may be produced according to the method (b) according to the present invention.

The amino compounds necessary as reaction components when performing the method (c, variation α) according to the present invention are generally defined by the formula (V). In this formula, R⁷ preferably represents hydrogen or alkyl having 1 to 4 carbon atoms, especially preferably hydrogen, methyl or ethyl.

Acid addition salts also come into consideration as reaction components, preferably hydrogen chloride addition salts of amino compounds of the formula (V).

Both the amino compounds of the formula (V) and also their acid addition salts are known or may be produced according to known methods.

All typical inert, organic solvents come into consideration as the diluent when performing the method (c, variation α) according to the present invention. Alcohols are preferably usable, such as methanol, ethanol, n-propanol or isopropanol.

All reaction accelerators typical for reactions of this type come into consideration as catalysts when performing the method (c, variation α) according to the present invention. Acidic or basic catalysts are preferably usable, such as the weakly basic ion exchanger commercially available under the name Amberlyst A-21®.

The reaction temperatures may be varied within a specific range when performing the method (c, variation α) according to the present invention. In general, one operates at temperatures between 0° C. and 80° C., preferably between 10° C. and 60° C.

When performing the method (c, variation α) according to the present invention, generally an equivalent quantity or an excess, preferably between 1.1 and 1.5 mol of amino compound of the formula (V) or an acid addition salt thereof is used for 1 mol of pyrazolopyrimidine of the formula (Ib). The workup is performed according to typical methods. In general, the reaction mixture is optionally filtered, then concentrated and purified.

The triphenylphosphonium salts necessary as reaction components when performing the method (c, variation β) according to the present invention are generally defined by the formula (VI). In this formula, Ph represents phenyl. R⁸ preferably represents hydrogen or alkyl having 1 to 4 carbon atoms, the alkyl residues being able to be substituted by carboxyl, methoxycarbonyl or ethoxycarbonyl. R⁸ especially preferably represents hydrogen, methyl or ethyl, the two latter residues being able to be substituted by carboxyl, methoxycarbonyl or ethoxycarbonyl.

The triphenylphosphonium salts of the formula (VI) are known or may be produced according to known methods.

All deprotonation agents typical for Wittig reactions of this type come into consideration as bases when performing the method (c, variation β) according to the present invention. Butyl lithium is preferably usable.

All organic solvents typical for Wittig reactions of this type come into consideration as the diluent when performing the method (c, variation β) according to the present invention. Ethers, such as dioxane or tetrahydrofuran, are preferably usable.

The reaction temperatures may be varied within a specific range when performing the method (c, variation β) according to the present invention. In general, one operates at temperatures between −78° C. and +30° C.

When performing the method (c, variation β) according to the present invention, an equivalent quantity or even an excess of triphenylphosphonium salt of the formula (VI) and an equivalent quantity or even an excess of base is used for 1 mol of pyrazolopyrimidine of the formula (Ib). The workup is performed according to typical methods.

The alkylation agents necessary as reaction components when performing the method (c, variation γ) according to the present invention are generally defined by the formula (VI). In this formula, R⁹ preferably represents alkyl having 1 to 4 carbon atoms, especially preferably methyl or ethyl. X³ preferably represents chloride, bromide, iodide, or the residue R⁹—O—SO₂—O—, in which R⁹ has the meanings specified above.

The alkylation agents of the formula (VU) are known or may be produced according to known methods.

If one uses diisobutyl aluminum hydride as a reducing agent in the first step when performing the method (c, variation γ) according to the present invention, it is expedient to operate under the conditions which were already cited in connection with the description of the method (b, variation α) according to the present invention.

If one uses sodium borohydride as a reducing agent in the first step when performing the method (c, variation γ) according to the present invention, one generally uses alcohols, preferably methanol, ethanol or isopropanol, as a diluent.

During the reduction using sodium borohydride, the reaction temperatures may be varied within a specific range. In general, one operates at temperatures between 0° C. and 70° C., preferably between 0° C. and 50° C.

When performing the reduction using sodium borohydride, an equivalent quantity or even an excess of sodium borohydride is used for 1 mol of pyrazolopyrimidine of the formula (Ib). The workup is performed according to typical methods.

All typical acid binders come into consideration as bases when performing the second step of the method (c, variation γ) according to the present invention. Alkali metal hydrides, alcoholates and carbonates are preferably usable, such as sodium hydride, sodium methylate, potassium tert. butylate, sodium carbonate, potassium carbonate or lithium carbonate.

All typical inert organic solvents come into consideration as the diluent when performing the second step of the method (c, variation γ) according to the present invention. Ethers, such as dioxane or tetrahydrofuran, and additionally nitrites, such as acetonitrile, are preferably usable.

The temperatures may be varied within a large range when performing the second step of the method (c, variation γ) according to the present invention. In general, one operates at temperatures between 0° C. and 100° C., preferably between 20° C. and 80° C.

When performing the second step of the method (c, variation γ) according to the present invention, in general, 1 to 2 mol, preferably 1 to 1.5 mol of alkylation agent is used for 1 mol of pyrazolopyrimidine of the formula (Ic). The workup is again performed according to typical methods.

The pyrazolopyrimidines necessary as starting materials when performing the method (d) according to the present invention are generally described by the formula (Id). In this formula, R¹, R², R³, R⁴ and Hal preferably have those meanings which were already cited as preferred for these residues in connection with the description of the materials of the formula (I) according to the present invention. R¹⁰ preferably represents hydrogen or alkyl having 1 to 4 carbon atoms, especially preferably hydrogen, methyl, ethyl or propyl, the three latter residues cited being able to be substituted by carboxyl, methoxycarbonyl or ethoxycarbonyl.

The pyrazolopyrimidines of the formula (Id) are materials according to the present invention which may be produced according to the method (a) according to the present invention.

According to a special method variation, the pyrazolopyrimidines may be produced by reacting
k) pyrazolopyrimidines of the formula 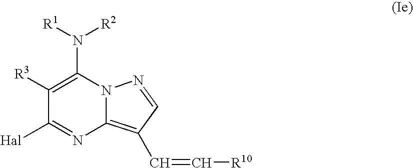
in which
R¹, R², R³, R⁴, R¹⁰ and Hal have the meanings specified above,
   with bromine in the presence of an inert, organic diluent, such as dichloromethane, trichloromethane or tetrachloromethane, at temperatures between −20° C. and +20°. The reaction components are preferably used in approximately equivalent quantities in this case. The workup is performed according to typical methods.

Preferably alkali metal alcoholates come into consideration as the strong bases when performing the method (d) according to the present invention, sodium methylate and potassium tert.-butylate being cited as examples.

All inert organic solvents typical for reactions of this type come into consideration as the diluent when performing the method (d) according to the present invention. Alcohols, such as methanol or ethanol, and nitrites, such as acetonitrile, are preferably usable.

The temperatures may be varied within a specific range when performing the method (d) according to the present invention. In general, one operates at temperatures between −10° C. and +80° C., preferably between 0° C. and 60° C.

When performing the method (d) according to the present invention, generally 2 to 3 equivalents or even a greater excess of strong base is used for 1 mol of pyrazolopyrimidine of the formula (Id). The workup is again performed according to typical methods.

The pyrazolopyrimidines necessary as starting materials when performing the method (e) according to the present invention are generally defined by the formula (VE). In this formula, R¹, R², R³, R⁴ and Hal preferably have those meanings which were already cited as preferred for these residues in connection with the description of the materials according to the present invention of the formula (I).

The pyrazolopyrimidines of the formula (VIII) are known or may be produced according to known methods (vgl. PCT/EP 03/05 159).

The acyl derivatives necessary as reaction components when performing the method (e) are generally defined by the formula (IX). In this formula R¹¹ preferably represents alkyl having 1 to 4 carbon atoms, especially preferably methyl, ethyl or n-propyl. X⁴ preferably represents chloride or a residue of the formula 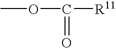
in which R¹¹ again has the previously specified meaning.

The acyl derivatives of the formula (IX) are known or may be produced according to known methods.

All reaction accelerators typical for Friedel-Crafts reactions of this type come into consideration as catalysts when performing the method (e) according to the present invention. Metal chlorides, such as aluminum trichloride or iron(III) chloride are preferably usable.

All inert organic solvents typical for reactions of this type come into consideration as the diluent when performing the method (e) according to the present invention. Ethers, such as diethylether, dioxane or tetrahydrofuran, are preferably usable.

The temperatures may be varied within a specific range when performing the method (e) according to the present invention. In general, one operates at temperatures between −20° C. and +20° C., preferably between −10° C. and +10° C.

When performing the method (e) according to the present invention, generally one uses 2 to 5 mol of acyl derivative of the formula (IX) and an appropriate quantity of catalyst for 1 mol of pyrazolopyrimidine of the formula (VIII). The workup is performed according to typical methods.

All solvents typical for halogenations of this type come into consideration as the diluent when performing the method (f) according to the present invention. Halogenated aliphatic or aromatic hydrocarbons, such as chlorobenzene, are preferably usable. However, the halogenation agent itself may function as the diluent, e.g., phosphorus oxychloride or a mixture of halogenation agents.

The temperatures may also be varied in a large range when performing the method (f). In general, one operates at temperatures between 0° C. and 150° C., preferably between 110° C. and 120° C.

When performing the method (f), hydroxypyrazolopyrimidine of the formula (X) is generally reacted with an excess of halogenation agent. The workup is performed according to typical methods.

All inert organic solvents typical for reactions of this type come into consideration as the diluent when performing the method (h). Alcohols are preferably usable, such as methanol, ethanol, n-propanol, i-propanol, n-butanol and tert.-butanol.

All inorganic and organic bases typical for reactions of this type come into consideration as the acid binder when performing the method (h). Tertiary amines, such as tributylamine or pyridine, are preferably usable. Amine used in excess may also function as a diluent.

The temperatures may be varied in a large range when performing the method (h). In general, one operates at temperatures between 20° C. and 200° C., preferably between 50° C. and 180° C.

When performing the method (h), heterocyclyl malonic esters of the formula (XII) and aminopyrazole of the formula (XIII) are generally reacted in equivalent quantities. However, it is also possible to use one or the other component in excess. The workup is performed according to typical methods.

All typical inert organic solvents come into consideration as the diluent when performing the methods (i) and (O) according to the present invention. Halogenated hydrocarbons, such as chlorobenzene, dichlorobenzene, dichloromethane, chloroform, tetrachloromethane, dichloroethane or trichlorethane; ethers, such as diethylether, diisopropylether, methyl-t-butylether, methyl-t-amylether, dioxane, tetrahydrofuran, 1,2-dimethoxyethane, 1,2-diethoxyethane or anisole; nitrites, such as acetonitrile, propionitrile, n- or i-butyronitrile or benzonitrile; amides, such as N,N-dimethylformamide, N,N-dimethylacetamide, N-methylformanilid, N-methylpyrrolidone or hexamethyl phosphoric triamide; sulphoxides, such as dimethylsulphoxide; sulphones, such as sulpholane; alcohols, such as methanol, ethanol, n- or i-propanol, n-, i-, sec- or tert-butanol, ethanediol, propane-1,2-diol, ethoxyethanol, methoxyethanol, diethylene glycol monomethylether, diethylene glycol monoethylether, their mixtures with water or even pure water are preferably usable.

The particular typical copper salts come into consideration as copper salts when performing the methods (i) and (j) according to the present invention. Copper(I) chloride or copper(I) bromide are preferably usable.

All inorganic or organic bases typical for reactions in this type come into consideration as acid acceptors when performing the methods (i) and (O) according to the present invention. Alkaline earth metal or alkali metal hydrides, hydroxides, amides, alcoholates, acetates, carbonates or hydrogen carbonates, such as sodium hydride, sodium amide, lithium diisopropylamide, sodium methylate, sodium ethylate, potassium tert.-butylate, sodium hydroxide, potassium hydroxide, sodium acetate, potassium acetate, calcium acetate, sodium carbonate, potassium carbonate, potassium hydrogen carbonate and sodium hydrogen carbonate, and additionally ammonium compounds such as ammonium hydroxide, ammonium acetate and ammonium carbonate, as well as tertiary amines, such as trimethylamine, triethylamine, tributylamine, N,N-dimethylaniline, N,N-dimethyl-benzylamine, pyridine, N-methylpiperidine, N-methylmorpholine, N,N-dimethylaminopyridine, diazabicyclooctane (DABCO), diazabicyclononene (DBN) or diazabicycloundecene (DBU) are preferably usable.

The reaction temperatures may be varied in a wide range when performing the methods (i) and (1) according to the present invention. In general, one operates at temperatures between 0° C. and 150° C., preferably at temperatures between 0° C. and 80° C.

When performing the method (i) according to the present invention, generally 1 to 15 mol, preferably 1.3 to 8 mol of malonic ester of the formula (XV) is used for 1 mol of halopyridine of the formula (XIV). The workup is performed according to typical methods.

When performing the method (j) according to the present invention, generally 1 to 15 mol, preferably 1.3 to 8 mol of malonic ester of the formula (XV) is used for 1 mol of halopyrimidine of the formula (XVI). The workup is again performed according to typical methods.

The methods according to the present invention are generally performed at atmospheric pressure. However, it is also possible to work at elevated pressure.

The materials according to the present invention have a strong microbicidal effect and may be used for combating undesired micro-organisms, such as fungi and bacteria, in plant protection, and in material protection.

Fungicides may be used in plant protection for combating Plasmodiophoromycetes, Oomycetes, Chytridiomycetes, Zygomycetes, Ascomycetes, Basidiomycetes and Deuteromycetes.

Bactericides may be used in plant protection for combating Pseudomonadaceae, Rhizobiaceae, Enterobacteriaceae, Corynebacteriaceae and Streptomycetaceae.

Some pathogens of fungal and bacterial diseases, which fall under the generic terms listed above, will be listed as examples, but not as restrictions:

Xanthomonas species, such as Xanthomonas campestris pv. oryzae;

Pseudomonas species, such as Pseudomonas syringae pv. lachrymans;

Erwinia species, such as Erwinia amylovora;

Pythium species, such as Pythium ultimum;

Phytophthora species, such as Phytophthora infestans;

Pseudoperonospora species, such as Pseudoperonospora humuli or

Pseudoperonospora cubensis;

Plasmopara species, such as Plasmopara viticola;

Bremia species, such as Bremia lactucae;

Peronospora species, such as Peronospora pisi or P. brassicae;

Erysiphe species, such as Erysiphe graminis;

Sphaerotheca species, such as Sphaerotheca fuliginea;

Podosphaera species, such as Podosphaera leucotricha;

Venturia species, such as Venturia inaequalis;

Pyrenophora species, such as Pyrenophora teres or P. graminea

(conidia form: Drechslera, syn: Helminthosporium);

Cochliobolus species, such as Cochliobolus sativus

(conidia form: Drechslera, syn: Helminthosporium);

Uromyces species, such as Uromyces appendiculatus;

Puccinia species, such as Puccinia recondita;

Sclerotinia species, such as Scierotinia sclerotiorum;

Tilletia species, such as Tilletia caries;

Ustilago species, such as Ustilago nuda or Ustilago avenae;

Pellicularia species, such as Pellicularia sasakii;

Pyricularia species, such as Pyricularia oryzae;

Fusarium species, such as Fusarium culmorum;

Botrytis species, such as Botrytis cinerea;

Septoria species, such as Septoria nodorum;

Leptosphaeria species, such as Leptosphaeria nodorum;

Cercospora species, such as Cercospora canescens;

Alternaria species, such as Alternaria brassicae;

Pseudocercosporella species, such as Pseudocercosporella herpotrichoides.

The active ingredients according to the present invention also have a very good strengthening effect in plants. They are therefore suitable for mobilizing plant defences against infection by undesired micro-organisms.

Plant-strengthening (resistance-inducing) materials are to be understood in the present context as those substances which are capable of stimulating the defence system of plants in such a way that, upon subsequent inoculation with undesired micro-organisms, the treated plants unfold extensive resistance to these micro-organisms.

In the present case, undesired micro-organisms are to be understood as phytopathogenic fungi, bacteria, and viruses. The materials according to the present invention may thus be used for protecting plants against infection by the pathogens cited within a certain period of time after treatment. The period of time within which this protection is provided generally extends from 1 to 10 days, preferably 1 to 7 days after the treatment of the plants with the active ingredients.

The good phytotolerance of the active ingredients in the concentrations necessary for combating plant diseases allows treatment of aboveground plant parts, of plants and seeds, and of the soil.

In this case, the active ingredients according to the present invention may be used especially successfully for combating grain diseases, such as Erysiphe species, and of diseases in wine, fruit, and vegetable farming, such as Botrytis, Venturia, Sphaerotheca and Podosphaera species.

The active ingredients according to the present invention are also suitable for increasing the harvest yield. They also have low toxicity and good phytotolerance.

The active ingredients according to the present invention may optionally also be used in specific concentrations and applied quantities as herbicides, to influence plant growth, and to combat animal pests. They may also be used as intermediate and precursor products for synthesizing further active ingredients if necessary.

According to the present invention, all plants and plant parts may be treated. Plants are understood in this case as all plants and plant populations, such as desired and undesired wild plants or cultured plants (including naturally occurring cultured plants). Cultured plants may be plants which are obtained through conventional cultivation and optimization methods or through methods of biotechnology and genetic engineering or combinations of these methods, including transgenic plants and including plant species which may or may not be protected by species protection rights. Plant parts are to be understood as all aboveground and below ground parts and organs of the plants, such as sprouts, leaves, flowers, and roots, for example, leaves, needles, stakes, stems, flowers, fruits, and seeds, as well as roots, bulbs, and rhizomes being listed. The plant parts also include hereditary material as well as vegetative and generative propagation material, such as slips, bulbs, rhizomes, cuttings, and seeds.

The treatment of the plants and plant parts according to the present invention using the active ingredients is performed directly or through the effect on their environment, living space, or storage space according to the typical treatment methods, e.g., through dipping, spraying, vaporizing, misting, scattering, painting, and for propagation material, particularly for seeds, also through single-layer or multilayered enveloping.

In material protection, the materials according to the present invention may be used for protecting technical materials against infection and destruction by undesired micro-organisms.

Technical materials are to be understood in the present context as inanimate materials which have been prepared for use in technology. For example, technical materials which may be protected by active ingredients according to the present invention from microbial change or destruction are adhesives, glues, paper and cardboard, textiles, leather, wood, paints and plastic articles, coolants, and other materials which may be infected or destroyed by micro-organisms. Parts of production facilities, such as coolant water loops, which may be impaired by reproduction of micro-organisms, are also cited in the scope of the materials to be protected. Preferably, adhesives, glues, paper and cardboard, leather, wood, paints, coolants, and thermal transfer fluids are cited as technical materials in the scope of the present invention, especially preferably wood.

For example, bacteria, fungi, yeasts, algae, and slime organisms are cited as micro-organisms which may cause degradation or change of the technical materials. Preferably, the active ingredients according to the present invention act against fungi, particularly mold fungi, wood-staining and wood-destroying fungi (Basidiomycetes), and against slime organisms and algae.

Micro-organisms of the following species are cited as examples:

Alternaria, such as Alternaria tenuis,

Aspergillus, such as Aspergillus niger,

Chaetomium, such as Chaetomium globosum,

Coniophora, such as Coniophora puetana,

Lentinus, such as Lentinus tigrinus,

Penicillium, such as Penicillium glaucum,

Polyporus, such as Polyporus versicolor,

Aureobasidium, such as Aureobasidium pullulans,

Scierophoma, such as Sclerophoma pityophila,

Trichoderma, such as Trichoderma viride,

Escherichia, such as Escherichia coli,

Pseudomonas, such as Pseudomonas aeruginosa,

Staphylococcus, such as Staphylococcus aureus.

As a function of their particular physical and/or chemical properties, the active ingredients may be converted into the typical formulations, such as solvents, emulsions, suspensions, powders, foams, pastes, granules, aerosols, extremely fine encapsulations in polymer materials, and into envelope compounds for seeds, as well as ULV cold and hot mist formulations.

These formulations are produced in ways known per se, e.g., by mixing the active ingredients with extenders, i.e., liquid solvents, liquefied gases under pressure, and/or solid carrier materials, optionally using surfactants, i.e., and also emulsifiers and/or dispersing agents and/or foam-producing agents. If water is used as an extender, organic solvents may also be used as an auxiliary solvents, for example. The following solvents essentially come into consideration as the liquid solvent: aromatics, such as xylene, toluene or alkylnaphthaline, chlorinated aromatics or chlorinated aliphatic hydrocarbons, such as chlorobenzene, chloroethylene or methylene chloride, aliphatic hydrocarbons, such as cyclohexane, or paraffins, such as petroleum fractions, alcohols, such as butanol or glycol as well as their ethers and esters, ketones, such as acetone, methylethylketone, methylisobutylketone or cyclohexanone, strongly polar solvents, such as dimethylformamide and dimethylsulphoxide, as well as water. Liquefied gaseous extenders or carriers are those liquids which are gaseous at normal temperature and under normal pressure, such as aerosol propellant gases, such as halogenated hydrocarbons as well as butane, propane, nitrogen and carbon dioxide. The following materials come into consideration as solid carriers: for example, natural rock flours, such as kaolin, aluminum oxide, talcum, chalk, quartz, attapulgite, montmorillonite or diatomaceous earths and synthetic rock flours, such as highly dispersed silicic acid, aluminum oxide and silicates. The following materials come into consideration as solid carriers for granules: for example, broken and fractionated natural stones such as calcite, pumice, marble, sepiolite, dolomite, as well as synthetic granulates made of inorganic and organic flours and granulates made of organic material like sawdust, coconut shells, maize cobs, and tobacco stalks. The following materials come into consideration as emulsifiers and/or foam-producing agents: for example, non-ionogenic and anionic emulsifiers, such as polyoxyethylene fatty acid esters, polyoxyethylene fatty alcohol ethers, e.g., alkylaryl polyglycolethers, alkyl sulphonates, alkyl sulphates, aryl sulphonates and protein hydrolysates. The following materials come into consideration as dispersing agents: e.g., lignin sulphite waste liquors and methyl cellulose.

Adhesives such as carboxymethylcellulose, natural and synthetic powdered, grainy, or latex polymers may be used in the formulations, such as gum arabic, polyvinylalcohol, polyvinylacetate, as well as natural phospholipids, such as kephalins and lecithins, and synthetic phospholipids. Further additives may be mineral and vegetable oils.

Coloring agents such as inorganic pigments, e.g., iron oxide, titanium oxide, ferrocyanide blue, and organic coloring agents such as alizarin, azo and metal phthalocyanine coloring agents and trace nutrients, such as salts of iron, manganese, boron, copper, cobalt, molybdenum, and zinc may be used.

The formulations generally contain between 0.1 and 95 percent by weight active ingredient, preferably between 0.5 and 90%.

The active ingredients according to the present invention may also be used per se or in their formulations with known fungicides, bactericides, acaricides, nematicides or insecticides, in order to thus broaden the activity spectrum or avoid the development of resistance, for example. In many cases, synergistic effects are achieved in this case, i.e., the effectiveness of the mixture is greater than the effectiveness of the individual components.

The following compounds come into consideration as mixing partners, for example:

Fungicides:

2-phenylphenol; 8-hydroxychinolinsulphat;

acibenzenear-S-methyl; aldimorph; amidoflumet; ampropylfos; ampropylfos-potassium; andoprim; anilazine; azaconazole; azoxystrobin;

benalaxyl; benodanil; benomyl; benthiavalicarb isopropyl; benzamacril; benzamacril-isobutyl; bilanafos; binapacryl; biphenyl; bitertanol; blasticidin-s; bromuconazole; bupirimate; buthiobate; butylamine;

calcium polysulphide; capsimycin; captafol; captan; carbendazim; carboxin; carpropamid; carvone; chinomethionat; chlobenthiazone; chlorofenazole; chloroneb; chlorothalonil; chlozolinate; clozylacon; cyazofamid; cyflufena mid; cymoxanil; cyproconazole; cyprodinil; cyprofuram;

Dagger G; debacarb; dichlofluanid; dichlone; dichlorophen; diclocymet; diclomezine; dicloran; diethofencarb; difenoconazole; diflumetorim; dimethirimol; dimethomorph; dimoxystrobin; diniconazole; diniconazole-m; dinocap; diphenylamine; dipyrithione; ditalimfos; dithianon; dodine; drazoxolon;

edifenphos; epoxiconazole; ethaboxam; ethirimol; etridiazole;

famoxadone; fenamidone; fenapanil; fenarimol; fenbuconazole; fenfuram; fenhexamid; fenitropan; fenoxanil; fenpiclonil; fenpropidin; fenpropimorph; ferbam; fluazinam; flubenzimine; fludioxonil; flumetover; flumorph; fluoromide; fluoxastrobin; fluquinconazole; flurprimidol; flusilazole; flusulphamide; flutolanil; flutriafol; folpet; fosetyl-Al; fosetyl sodium; fuberidazole; furalaxyl; furametpyr; furcarbanil; furmecyclox;

guazatine;

hexachlorobenzene; hexaconazole; hymexazol;

imazalil; imibenconazole; iminoctadine triacetate; iminoctadine tris(albesil); iodocarb; ipconazole; iprobenfos; iprodione; iprovalicarb; irumamycin; isoprothiolane; isovaledione;

kasugamycin; kresoxim-methyl;

mancozeb; maneb; meferimzone; mepanipyrim; mepronil; metalaxyl; metalaxyl-m; metconazole; methasulphocarb; methfuroxam; metiram; metominostrobin; metsulphovax; mildiomycin; myclobutanil; myclozolin;

natamycin; nicobifen; nitrothal-isopropyl; noviflumuron; nuarimol;

ofurace; orysastrobin; oxadixyl; oxolinic acid; oxpoconazole; oxycarboxin; oxyfenthiin;

paclobutrazol; pefurazoate; penconazole; pencycuron; phosdiphen; phthalide; picoxystrobin; piperalin; polyoxins; polyoxorim; probenazole; prochloraz; procymidone; propamocarb; propanosine-sodium; propiconazole; propineb; proquinazid; prothioconazole; pyraclostrobin; pyrazophos; pyrifenox; pyrimethanil; pyroquilon; pyroxyfur; pyrrolnitrine;

quinconazole; quinoxyfen; quintozene;

simeconazole; spiroxamine; sulphur;

tebuconazole; tecloftalam; tecnazene; tetcyclacis; tetraconazole; thiabendazole; thicyofen; thifluzamide; thiophanate-methyl; thiram; tioxymid; tolclofos-methyl; tolylfluanid; triadimefon; triadimenol; triazbutil; triazoxide; tricyclamide; tricyclazole; tridemorph; trifloxystrobin; triflumizole; triforine; triticonazole;

uniconazole;

validamycin a; vinclozolin;

zineb; ziram; zoxamide;

(2S)—N-[2-[4-[[3-(4-chlorophenyl)-2-propinyl]oxy]-3-methoxyphenyl]ethyl]-3-methyl-2-[(methylsulphonyl)amino]-butanamide;

1-(1-naphthalenyl)-1H-pyrrol-2,5-dion;

2,3,5,6-tetrachlor-4-(methylsulphonyl)-pyridine;

2-amino-4-methyl-n-phenyl-5-thiazolcarboxamide;

2-chloro-n-(2,3-dihydro-1,1,3-trimethyl-1H-inden-4-yl)-3-pyridincarboxamide;

3,4,5-trichloro-2,6-pyridindicarbonitrile;

actinovate;

cis-1-(4-chlorophenyl)-2-(1H-1,2,4-triazol-1-yl)-cycloheptanol;

methyl 1-(2,3-dihydro-2,2-dimethyl-1H-inden-1-yl)-1H-imidazol-5-carboxylate;

monopotassium carbonate;

n-(6-methoxy-3-pyridinyl)-cyclopropancarboxamide;

sodium tetrathiocarbonate;

as well as copper salts and preparations, such as Bordeaux mixture; copper hydroxide; copper naphthenate; copper oxychloride; copper sulphate; cufraneb; copper oxide; mancopper; oxine copper.

Bactericides:

bronopol, dichlorophen, nitrapyrin, nickel dimethyldithiocarbamate, kasugamycin, octhilinon, furan carboxylic acid, oxytetracyclin, probenazol, streptomycin, tecloftalam, copper sulphate and other copper preparations.

Insecticides/Acaricides/Nematicides:

abamectin, ABG-9008, acephate, acequinocyl, acetamiprid, acetoprole, acrinathrin, AKD-1022, AKD-3059, AKD-3088, alanycarb, aldicarb, aldoxycarb, allethrin, allethrin 1R-isomers, alpha-cypermethrin (alpharnethrin), amidoflumet, aminocarb, amitraz, avermectin, AZ-60541, azadirachtin, azamethiphos, azinphos-methyl, azinphos-ethyl, azocyclotin,

Bacillus popilliae, Bacillus sphaericus, Bacillus subtilis, Bacillus thuringiensis, Bacillus thuringiensis strain EG-2348, Bacillus thuringiensis strain GC-91, Bacillus thuringiensis strain NCTC-11821, baculoviruses, Beauveria bassiana, Beauveria tenella, bendiocarb, benfuracarb, bensultap, benzoximate, beta-cyfluthrin, beta-cypermethrin, bifenazate, bifenthrin, binapacryl, bioallethrin, bioallethrin-S-cyclopentyl-isomer, bioethanomethrin, biopermethrin, bioresmethrin, bistrifluron, BPMC, brofenprox, bromophos ethyl, bromopropylate, bromfenvinfos (methyl), BTG-504, BTG-505, bufencarb, buprofezin, butathiofos, butocarboxim, butoxycarboxim, butylpyridaben,

cadusafos, camphechlor, carbaryl, carbofuran, carbophenothion, carbosulphan, cartap, CGA-50439, chinomethionat, chlordane, chlordimeform, chloethocarb, chlorethoxyfos, chlorfenapyr, chlorfenvinphos, chlorfluazuron, chlormephos, chlorobenzilate, chloropicrin, chlorproxyfen, chlorpyrifos methyl, chlorpyrifos (ethyl), chlovaporthrin, chromafenozide, cis-cypermethrin, cis-resmethrin, cis-permethrin, clocythrin, cloethocarb, clofentezine, clothianidin, clothiazoben, codlemone, coumaphos, cyanofenphos, cyanophos, cycloprene, cycloprothrin, cydia pomonella, cyfluthrin, cyhalothrin, cyhexatin, cypermethrin, cyphenothrin (1R-trans-isomer), cyromazine,

DDT, deltamethrin, demeton-S-methyl, demeton-S-methylsulphon, diafenthiuron, dialifos, diazinon, dichlofenthion, dichlorvos, dicofol, dicrotophos, dicyclanil, diflubenzuron, dimethoate, dimethylvinphos, dinobuton, dinocap, dinotefuran, diofenolan, disulphoton, docusat-sodium, dofenapyn, DOWCO-439,

eflusilanate, emamectin, emamectin-benzoate, empenthrin (1R-isomer), endosulphan, Entomopthora spp., EPN, esfenvalerate, ethiofencarb, ethiprole, ethion, ethoprophos, etofenprox, etoxazole, etrimfos,

famphur, fenamiphos, fenazaquin, fenbutatin oxide, fenfluthrin, fenitrothion, fenobucarb, fenothiocarb, fenoxacrim, fenoxycarb, fenpropathrin, fenpyrad, fenpyrithrin, fenpyroximate, fensulphothion, fenthion, fentrifanil, fenvalerate, fipronil, flonicamid, fluacrypyrim, fluazuron, flubenzimine, flubrocythrinate, flucycloxuron, flucythrinate, flufenerim, flufenoxuron, flufenprox, flumethrin, flupyrazofos, flutenzin (flufenzine), fluvalinate, fonofos, formetanate, formothion, fosmethilan, fosthiazate, fubfenprox (fluproxyfen), furathiocarb,

gamma HCH, gossyplure, grandlure, granulose viruses,

halfenprox, halofenozide, HCH, HCN-801, heptenophos, hexaflumuron, hexythiazox, hydramethylnone, hydroprene,

IKA-2002, imidacloprid, imiprothrin, indoxacarb, iodofenphos, iprobenfos, isazofos, isofenphos, isoprocarb, isoxathion, ivermectin,

japonilure,

kadethrin, nuclear polyhedrosis viruses, kinoprene,

lambda cyhalothrin, lindane, lufenuron,

malathion, mecarbam, mesulphenfos, metaldehyd, metam-sodium, methacrifos, methamidophos, metharhizium anisopliae, metharhizium flavoviride, methidathion, methiocarb, methomyl, methoprene, methoxychlor, methoxyfenozide, metolcarb, metoxadiazone, mevinphos, milbemectin, milbemycin, MKI-245, MON-45700, monocrotophos, moxidectin, MTI-800,

naled, NC-104, NC-170, NC-184, NC-194, NC-196, niclosamide, nicotine, nitenpyram, nithiazine, NNI-0001, NNI-0101, NNI-0250, NNI-9768, novaluron, noviflumuron,

OK-5101, OK-5201, OK-9601, OK-9602, OK-9701, OK-9802, omethoate, oxamyl, oxydemeton-methyl,

Paecilomyces fumosoroseus, parathion methyl, parathion (ethyl), permethrin (cis-, trans-), petroleum, PH-6045, phenothrin (1R-trans isomer), phenthoate, phorate, phosalone, phosmet, phosphamidon, phosphocarb, phoxim, piperonyl butoxide, pirimicarb, pirimiphos methyl, pirimiphos ethyl, prallethrin, profenofos, promecarb, propaphos, propargite, propetamphos, propoxur, prothiofos, prothoate, protrifenbute, pymetrozine, pyraclofos, pyresmethrin, pyrethrum, pyridaben, pyridalyl, pyridaphenthion, pyridathion, pyrimidifen, pyriproxyfen,

quinalphos,

resmethrin, RH-5849, ribavirin, RU-12457, RU-15525,

S-421, S-1833, salithion, sebufos, SI-0009, silafluofen, spinosad, spirodiclofen, spiromesifen, sulphluramid, sulphotep, suiprofos, SZI-121,

tau-fluvalinate, tebufenozide, tebufenpyrad, tebupirimfos, teflubenzuron, tefluthrin, temephos, temivinphos, terbam, terbufos, tetrachlorvinphos, tetradifon, tetramethrin, tetramethrin (1R isomer), tetrasul, theta-cypermethrin, thiacloprid, thiamethoxam, thiapronil, thiatriphos, thiocyclam hydrogen oxalate, thiodicarb, thiofanox, thiometon, thiosultap sodium, thuringiensin, tolfenpyrad, tralocythrin, tralomethrin, transfluthrin, triarathene, triazamate, triazophos, triazuron, trichlophenidine, trichlorfon, triflumuron, trimethacarb,

vamidothion, vaniliprole, verbutin, Verticillium lecanii,

WL-108477, WL-40027,

YI-5201, YI-5301, YI-5302,

XMC, xylylcarb,

ZA-3274, zeta-cypermethrin, zolaprofos, ZXI-8901,

the compound 3-methyl-phenyl-propylcarbamate (Tsumacide Z),

the compound 3-(5-chloro-3-pyridinyl)-8-(2,2,2-trifluorethyl)-8-azabicyclo[3.2.1]octane-3-carbonitrile (CAS-Reg.-No. 185982-80-3) and the corresponding 3-endo-isomers (CAS-Reg.-No. 185984-60-5) (cf. WO-96/37494, WO-98/25923),

as well as preparations which contain insecticidally active plant extracts, nematodes, fungi, or viruses.

A mixture with other known active ingredients, such as herbicides, or with fertilizers and growth regulators, safeners, and/or semiochemicals is also possible.

In addition, the compounds of the formula (I) according to the present invention also have very good antimycotic effect. They have a very broad antimycotic activity spectrum, particularly against dermatophytes and sprout fungi, mold and diphasic fungi (e.g., against Candida species such as Candida albicans, Candida glabrata) as well as Epidermophyton floccosum, Aspergillus species such as Aspergillus niger and Aspergillus fumigatus, Trichophyton species such as Trichophyton mentagrophytes, Microsporon species such as Microsporon canis und audouinii. The list of these fungi does not represent a restriction of the mycotic spectrum which may be contained, but rather only has explanatory character.

Furthermore, the compounds of the formula (I) according to the present invention are suitable for suppressing the growth of tumour cells in humans and mammals. This is based on an interaction of the compounds according to the present invention with tubulin and microtubules and through encouragement of microtubule polymerization.

For this purpose, an effective quantity of one or more compounds of the formula (I) or pharmaceutically compatible salts thereof may be administered.

The active ingredients may be applied as such, in the form of their formulations or the application forms prepared therefrom, such as ready-to-use solutions, suspensions, spray powders, pastes, soluble powders, dusting agents, and granules. The application is performed in the typical way, e.g., through pouring, spraying, scattering, dusting, foaming, painting, etc. Furthermore, it is possible to apply the active ingredients according to the ultralow volume method or inject the active ingredient preparation or the active ingredient itself into the soil. The seed of the plants may also be treated.

When using the active ingredients according to the present invention as fungicides, the applied quantities may be varied within a wide range depending on the type of application. When treating plant parts, the applied quantities of active ingredient are generally between 0.1 and 10,000 g/hectare, preferably between 10 and 1000 g/hectare. When seeds are treated, the applied quantities of active ingredient are generally between 0.001 and 50 g per kilogram of seed, preferably between 0.01 and 10 g per kilogram of seed. When treating the soil, the applied quantities of active ingredient are generally between 0.1 and 10,000 g/hectare, preferably between 1 and 5000 g/hectare.

As already noted above, all plants and their parts may be treated according to the present invention. In a preferred embodiment, types of plants and plant species occurring wild or obtained through conventional biological cultivation methods, such as breeding or protoplast fusion, as well as their parts, may be treated. In a further preferred embodiment, transgenic plants and plant species which were obtained through methods of genetic engineering, optionally in combination with conventional methods (genetically modified organisms) and their parts are treated. The term “parts” and/or “parts of plants” or “plant parts” was explained above.

According to the present invention, plants of the particular commercially available plant species or plant species in use are especially preferably treated. Plant species are understood as plants having new properties (“traits”), which may be cultivated both through conventional cultivation, through mutagenesis, or through recombinant DNA technologies. These may be species, breeds, biotypes, and genotypes.

Depending on the plant types and/or plant species, their location and growth conditions (soil, climate, vegetation period, nutrition), synergistic effects may also arise through the treatment according to the present invention. Thus, for example, lowered applied quantities and/or expansions of the activity spectrum and/or an amplification of the effect of the materials and agents usable according to the present invention, better plant growth, elevated tolerance to high or low temperatures, elevated tolerance drought or to water and/or soil salinity, elevated blooming performance, easier harvesting, acceleration of ripening, higher harvest yields, higher quality and/or higher nutritional value of the harvested products, greater storage capability and/or processability of the harvested products are possible, which exceed the actual effects to be expected.

The preferred transgenic (obtained through genetic engineering) plants and/or plant species to be treated according to the present invention include all plants which have obtained genetic material through genetic modification which provides these plants with especially advantageous valuable properties (“traits”). Examples of such properties are better plant growth, elevated tolerance to high or low temperatures, elevated tolerance to drought or to water and/or soil salinity, elevated blooming performance, easier harvesting, acceleration of ripeness, elevated harvest yields, greater storage capability and/or processability of the harvested products. Further and especially pronounced examples of such properties are elevated defence of the plants against animal and microbial pests, for example, against insects, mites, phytopathogenic fungi, bacteria, and/or viruses, as well as elevated tolerance of the plants to specific herbicidal active ingredients. Examples of transgenic plants include the important cultured plants, such as grains (wheat, rice), maize, soya, potatoes, cotton, tobacco, rapeseed, as well as fruit plants (having the fruits apples, pears, citrus fruits, and grapes), maize, soya, potatoes, cotton, tobacco, and rapeseed being noted in particular. The elevated defence of the plants to insects, arachnids, nematodes, and snails through toxins arising in the plants, particularly those which are generated in the plants by the genetic material of Bacillus thuringiensis (e.g., for example, by the genes CryIA(a), CryIA(b), CryIA(c), CryIIA, CryIIIA, CryIIIB2, Cry9c Cry2Ab, Cry3Bb and CryIF, as well as their combinations) are especially to be noted (referred to in the following as “Bt plants”). The elevated defences of plants against fungi, bacteria, and viruses through systemic acquired resistance (SAR), systemin, phytoalexines, elicitors, and resistance genes and correspondingly expressed proteins and toxins are also especially noted as properties (“traits”). The elevated tolerance of the plants to specific herbicidal active ingredients, such as imidazolinones, sulphonyl ureas, glyphosates, or phosphinotricine (e.g., “PAT” gene) is also especially to be noted. The particular genes which provide the desired properties (“traits”) may also occur in the transgenic plants in combination with one another. Examples of “Bt plants” are maize varieties, cotton varieties, soya varieties, and potato varieties which are distributed under the trade names YIELD GARD® (e.g., maize, cotton, soya), KnockOut® (e.g., maize), StarLink® (e.g., maize), Bollgard® (cotton), Nucoton® (cotton) and NewLeaf® (potato). Examples of plants tolerant to herbicides are maize varieties, cotton varieties and soya varieties, which are distributed under the trade names Roundup Ready® (tolerance to glyphosates, e.g., maize, cotton, soya), Liberty Link® (tolerance to phosphinotricine, e.g., rapeseed), IMI® (tolerance to imidazolinones), and STS® (tolerance to sulphonyl ureas, e.g., maize). The varieties (e.g., maize) of plants resistant to herbicides (conventionally cultivated for herbicide tolerance) distributed under the trade name Clearfield® are also noted. Of course, the statements also apply for plant varieties developed in the future and/or coming to market in the future having these genetic properties (“traits”) or those developed in the future.

The plants listed may be treated especially advantageously according to the present invention using the compounds of the general formula (I) and/or the active ingredient mixtures according to the present invention. The preferred ranges specified above for the active ingredients and/or mixtures also apply for the treatment of these plants. The plant treatment using the compounds and/or mixtures specially listed in the present text is especially noted.

The production and the use of the active ingredients according to the present invention is described in the following examples.

### PRODUCTION EXAMPLES

### Example 1

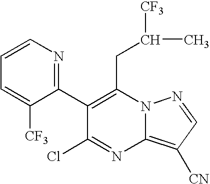 (Method a)

0.065 g (1.12 mmol) potassium fluoride is added to a solution of 0.2 g (0.56 mmol) 3-cyano-5,7-dichloro-6-(3-trifluoromethyl-pyridin-2-yl)-pyrazolo[1,5-a]pyrimidine in 10 ml acetonitrile, stirred 2 hours at 80° C. and subsequently cooled to 0° C. 0.13 g (1.17 mmol) (S)-2,2,2-trifluoro-isopropylamine is added to this solution and stirred 18 hours at 80° C. The reaction mixture is then cool to room temperature and stirred into 30 ml diluted hydrochloric acid. The mixture is extracted using dichloromethane, the organic phase is washed twice using water, dried over sodium sulphate, and concentrated under reduced pressure. The remaining residue is filtered using a mixture of petroleum ether/methyl tert.-butylether=15:1 via a short silica gel column. In this way, 0.15 g (58.5% of theoretical yield) of 3-cyano-5-chloro-6-(3-trifluoromethyl-pyridin-2-yl)-pyrazolo[1,5-a]-pyrimidin-N-[(1,S)-2,2,2-trifluoro-1-methylethyl]-amine is obtained.

HPLC: logP=3.14

The pyrazolopyrimidines of the formula

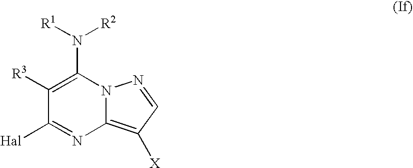

listed in the following Table I were also prepared according to the method specified above.

TABLE 1

| Ex. | | | | | | |
|---|---|---|---|---|---|---|
| No. | R¹ | R² | R³ | Hal | X | logP |
| 2 | 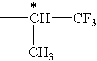 | H | 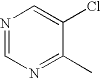 | Cl | —CN | 2.77 Isomer |
| 3 | 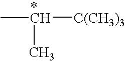 | H | 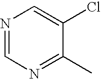 | Cl | —CN | 3.54 |
| 4 | 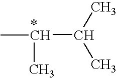 | H | 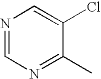 | Cl | —CN | 3.13 |
| 5 | 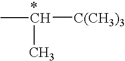 | H | 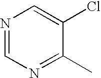 | Cl | —Cl | 4.36 |
| 6 | 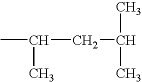 | H | 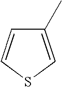 | Cl | —CN | 4.46 |
| 7 | 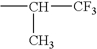 | H | 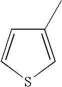 | Cl | —CN | 3.53 |
| 8 | —CH₂—CF₃ | H | 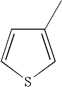 | Cl | —CN | 3.21 |
| 9 | 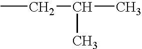 | H | 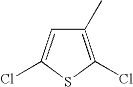 | Cl | —CN | 4.82 |
| 10 | 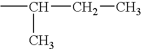 | H | 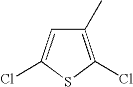 | Cl | —CN | 4.82 |
| 11 | 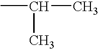 | H | 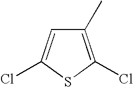 | Cl | —CN | 4.41 |
| 12 | 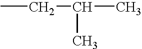 | —CH₃ | 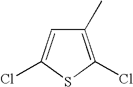 | Cl | —CN | 5.31 |
| 13 | 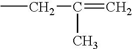 | —CH₃ | 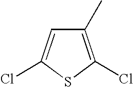 | Cl | —CN | 5.10 |
| 14 | —NH—CH₂—CH₂—CH₂—CH₂— | | 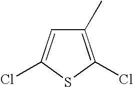 | —CN | 4.46 | |
| 15 | —CH₂—C(CH₃)₃ | H | 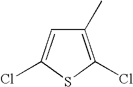 | Cl | —CN | 5.31 |
| 16 | 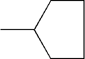 | H | 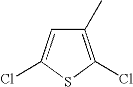 | Cl | —CN | 5.03 |
| 17 | —CH₂—CH₂—OCH₃ | H | 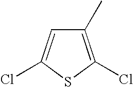 | Cl | —CN | 3.90 |
| 18 | —CH₂—CH₂—OCH₃ | —CH₃ | 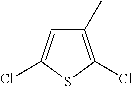 | Cl | —CN | 4.32 |
| 19 | —CH₂—CH₂—CN | —CH₃ | 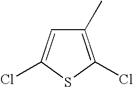 | Cl | —CN | 3.90 |
| 20 | 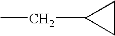 | —C₃H₇-n | 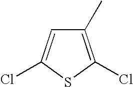 | Cl | —CN | 5.78 |
| 21 | —CH₂—CH₂—CH₂—CH₂— | | 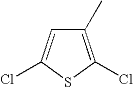 | Cl | —CN | 4.61 |
| 22 | 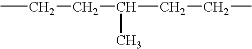 | | 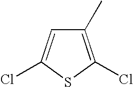 | Cl | —CN | 5.59 |
| 23 | —CH₂—CH₂—CH₂—CH₂—CH₂— | | 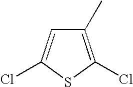 | Cl | —CN | 6.19 |
| 24 | 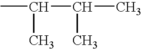 | H | 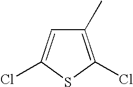 | Cl | —CN | 5.19 |
| 25 | 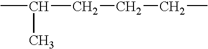 | | 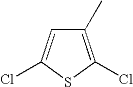 | Cl | —CN | 4.98 |
| 26 | 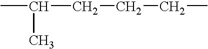 | | 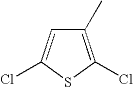 | Cl | —CN | 4.58 |
| 27 | 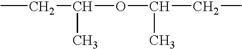 | | 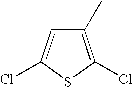 | Cl | —CN | 4.72 |
| 28 | —CH₂—CH₂—O—CH₂—CH₂— | | 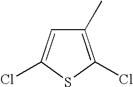 | Cl | —CN | 3.85 |
| 29 | 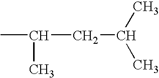 | H | 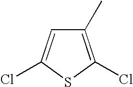 | Cl | —CN | 5.59 |
| 30 | —CH₂—CF₃ | H | 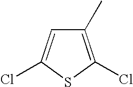 | Cl | —CN | 4.06 |
| 31 | 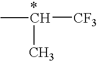 | H | 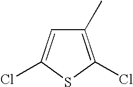 | Cl | —CN | 4.46 |
| 32 | —CH₂—C(CH₃)³ | H | 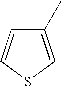 | Cl | —CN | 4.18 |
| 33 | 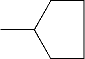 | H | 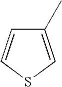 | Cl | —CN | 3.00 |
| 34 | 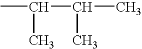 | H | 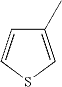 | Cl | —CN | 4.08 |
| 35 | 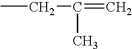 | —CH₃ | 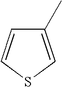 | Cl | —CN | 3.98 |
| 36 | 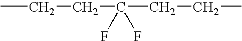 | | 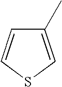 | Cl | —CN | 3.61 |
| 37 | 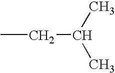 | H | 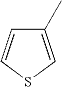 | Cl | —CN | 3.78 |
| 38 | 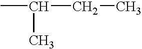 | H | 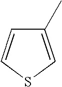 | Cl | —CN | 3.74 |
| 39 | 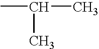 | H | 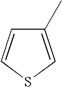 | Cl | —CN | 3.37 |
| 40 | 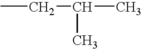 | —CH₃ | 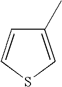 | Cl | —CN | 4.13 |
| 41 | —CH₂—CH₂—CH₂—CH₂— | | 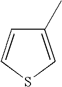 | Cl | —CN | 3.53 |
| 42 | 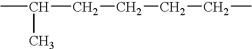 | | 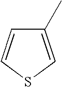 | Cl | —CN | 4.27 |
| 43 | 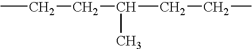 | | 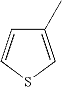 | Cl | —CN | 4.41 |
| 44 | —CH₂—CH₂—O—CH₂—CH₂— | | 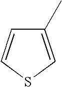 | Cl | —CN | 2.90 |
| 45 | —CH₂—CH₂—OCH₃ | H | 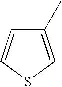 | Cl | —CN | 2.94 |
| 46 | 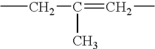 | —CH₂—CH₃ | 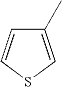 | Cl | —CN | 4.32 |
| 47 | 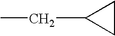 | —CH₂—CH₂—CH₃ | 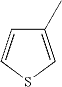 | Cl | —CN | 4.51 |
| 48 | 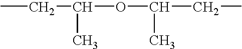 | | 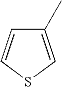 | Cl | —CN | 3.53 |
| 49 | —CH₂—CH₂—CH₂—CH₂—CH₂— | | 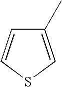 | Cl | —CN | 3.58 |
| 50 | 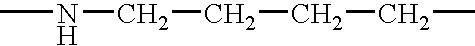 | | 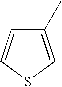 | Cl | —CN | 3.15 |
| 51 | —CH₂—CH₂—OCH₃ | —CH₃ | 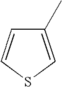 | Cl | —CN | 3.17 |
| 52 | —CH₂—CH₂—CN | —CH₂—CH₃ | 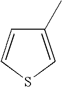 | Cl | —CN | 2.98 |
| 53 | 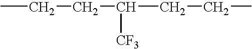 | | 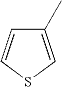 | Cl | —CN | 4.03 |
| 54 | —CH(CH₃)—CH₂—CH₂—CH₂— | | 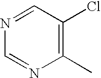 | Cl | —CHO | 2.44 |
| 55 | 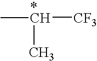 | H | 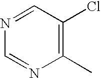 | Cl | —CHO | 2.46 |
| 56 | 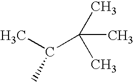 | H | 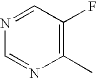 | Cl | —CN | 3.29 |
| 57 | 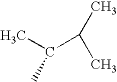 | H | 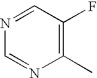 | Cl | —CN | 2.92 |
| 58 | 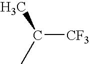 | H | 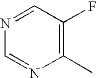 | Cl | —CN | 2.65 |
| 59 | 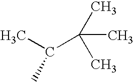 | H | 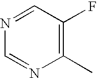 | Cl | Cl | 4.11 |
| 60 | 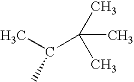 | H | 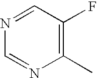 | Cl | H | 3.42 |
| 61 | 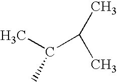 | H | 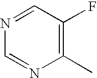 | Cl | H | 2.98 |
| 62 | 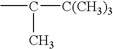 | H | 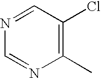 | Cl | —CHO | 3.12 |
| 63 | —CH₂CH₂CH(CH₃)—CH₂—CH₂— | | 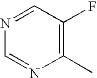 | Cl | —CN | 3.20 |
| 64 | 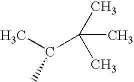 | H | 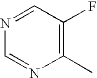 | Cl | —COOCH₃ | 3.15 |

Production of Precursor Products of the Formula (II):

### Example 65

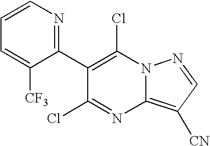 Method (f)

3.0 g (14.5 mmol) phosphorus pentachloride is added to a mixture of 5.8 g (18.1 mmol) 3-cyano-6-(3-trifluoromethyl-pyridin-2-yl)-pyrazolo[1,5-a]pyrimidin-5,7-diol and 22.15 g (144.5 mmol) phosphorus oxychloride at room temperature in 5 portions while stirring. The reaction mixture is heated 4 hours under reflux, then cooled to room temperature and concentrated under reduced pressure. The remaining residue is admixed with 100 ml water and then extracted three times using 100 ml dichloromethane each time. The combined organic phases are washed twice using 50 ml water each time, dried over sodium sulphate and concentrated under reduced pressure. The remaining residue is chromatographed using hexane/acetic ethylester=3:1 on silica gel. 0.88 g (14.8% of theoretical yield) of 3-cyano-5,7-dichloro-6-(3-trifluoromethyl-pyridin-2-yl)-pyrazolo-[1,5-a]pyrimidine is obtained in this way.

HPLC: logP=2.68

### Example 66

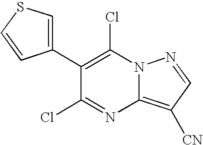

A mixture made of 2.0 g (10.74 mmol) 2-thienyl malonic acid and 1.16 g (10.74 mmol) 3-amino-4-cyano-pyrazole is admixed at room temperature within 2 minutes with 41.13 g (268 mmol) phosphorus oxychloride while stirring. The mixture is then heated for 18 hours to 90° C. and then cooled to room temperature. The reaction mixture is poured into 250 ml icewater, and the resulting suspension is stirred 1 hour. The mixture is suctioned and washed using 50 ml water. For further purification, the product is suspended in 50 ml cyclohexane/acetic ethylester=1:1 and briefly boiled, then cooled, suctioned via a short silica gel column and washed 8 times using 50 ml cyclohexane/acetic ethylester=1:1. The filtrate is dried over sodium sulphate and then filtered again. The filter residue is washed down using a little cyclohexane/acetic ethylester=1:1. All of the filtrate is concentrated under reduced pressure. 1.48 g (30.34% of theoretical yield) of 5,7-dichloro-3-cyano-6-(thien-3-yl)-pyrazolo[1,5-a]pyrimidine is obtained in the form of a solid.

### Example 67

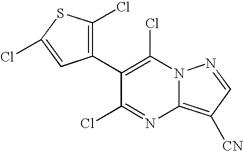

A chlorine gas stream is conducted for 2 hours into a solution of 7.5 g (25.41 mmol) 5,7-dichloro-3-cyano-6-(thien-3-yl)-pyrazolo[1,5-a]pyrimidine in 80 ml dichloromethane at temperatures between −5° C. and 0° C. The reaction mixture is then heated to room temperature and concentrated under reduced pressure. The remaining residue is absorbed using dichloromethane and suctioned off. 2.0 g of the desired product is obtained. The previously collected filtrate is chromatographed using cyclohexane/acetic ethylester=1:1 on silica gel after being concentrated. After concentration of the eluate, a further 3.5 g of the desired product is isolated. In this way, a total of 5.5 g (54.13% of theoretical yield) of 5,7-dichloro-3-cyano-(2,5-dichloro-thien-3-yl)-pyrazolo-[1,5-a]pyrimidine is obtained.

Production of Precursor Product of the Formula (X):

### Example 68

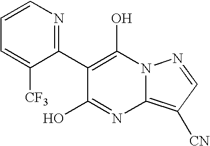 Method (h)

A mixture made of 4.1 g (14.8 mmol) 2-(3-trifluoromethyl-pyridin-2-yl)malonic dimethylester, 1.6 g (14.8 mmol) 3-amino-4-cyano-pyrazole and 3.02 g (16.3 mmol) tri-n-butylamine is heated for 2 hours to 180° C. while stirring. At the same time, the methanol arising during the reaction is continuously distilled off. Subsequently, the reaction mixture is cooled to room temperature. The separating tri-n-butylamine is decanted off, and the remaining mixture is distilled under reduced pressure. 5.8 g of a product is obtained which, according to HPLC, comprises 60% 3-cyano-6-(3-trifluoromethyl-pyridin-2-yl)-pyrazolo[1,5-a]pyrimidin-5,7-diol. The yield is accordingly calculated as 73.25% of theoretical yield. The product is used for further synthesis without additional purification.

HPLC: logP=0.29

Production of Precursor Product of the Formula (XII-a):

### Example 69

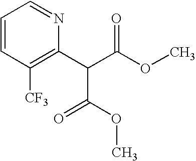 Method (i)

9 g (207 mmol) 60% sodium hydride suspension is suspended in 300 ml dioxane. 27.29 g (206.6 mmol) malonic dimethylester is dripped into this mixture at 55-60° C. and stirred for a further 30 minutes at the same temperature. After adding 8.18 g (82.63 mmol) copper(I) chloride, the mixture is heated to 80° C. and then 15 g (82.63 mmol) 2-chloro-3-trifluormethylpyridine is dripped in. The reaction mixture is now stirred 14 hours at 100° C. After the subsequent cooling to 15-20° C., concentrated hydrochloric acid is dripped in slowly until the mixture is acidic. 600 ml water and 300 ml dichloromethane are now added and insoluble components are filtered off. The organic phase is separated from the filtrate, dried over sodium sulphate, and concentrated under reduced pressure. The residue is chromatographed using hexane/acetic ester (4:1) on silica gel. 10.1 g (40% of theoretical yield) of 2-[3-trifluoromethyl]-pyrimidin-2-yl)malonic dimethylester is obtained.

HPLC: logP=2.05

Production of Precursor Product of the Formula (XII-b):

### Example 70

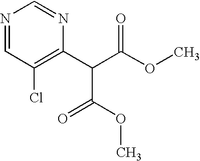 Method (j)

2.6 g (65.4 mmol) 60% sodium hydride suspension is suspended in 100 ml tetrahydrofuran. 6.9 g (52.4 mmol) malonic dimethylester is added at 0° C. and the mixture is stirred for 0.5 hours at the same temperature. A solution of 6.5 g (43.63 mmol) 4,5-dichloropyrimidine in 50 ml tetrahydrofuran is then dripped in and the mixture is stirred a further 3 hours at room temperature. 150 ml 1 N hydrochloric acid is then slowly dripped in and the mixture is then extracted using 100 ml dichloromethane. The organic phase is separated off, dried over sodium sulphate, and concentrated under reduced pressure. The residue is chromatographed on silica gel using methyl-t-butylether/petroleum ether (1:9). 7 g (65.6% of theoretical yield) of 2-(5-chloro-4-pyrimidin-2-yl)malonic dimethylester is obtained.

HPLC: logP=1.33

### Example 71

### Production of 4,5-dichlorpyrimidine

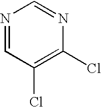

1.6 ml dimethylamine is added to a solution of 112.5 g (673.7 mmol) 5-chloro-6-oxo-1,6-dihydropyrimidin-1-ium chloride in 630 ml phosphorus oxychloride and heated for 3 hours under reflux. The excess phosphorus oxychloride is then distilled off under reduced pressure. After cooling, the residue is poured onto 1.5 l icewater, extracted using 500 ml dichloromethane, the organic phase is dried over sodium sulphate and concentrated under reduced pressure. 72.3 g (66.3% of theoretical yield) 4,5-dichloropyrimidine is obtained.

HPLC: logP=1.35

### Example 72

### Production of 5-chloro-6-oxo-1,6-dihydropyrimidin-1-ium chloride

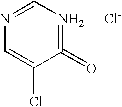

6.5 g (40 mmol) iron(III) chloride is added to a solution of 77 g (0.8 mol) 4(3H)-pyrimidinone in 770 ml glacial acetic acid and 113.6 g (1.6 mol) chlorine is introduced within 2 hours at 40-45° C. The reaction mixture is cooled to 15° C., the resulting solid product is suctioned off and washed using ether. 112.5 g (84% of theoretical yield) 5-chloro-6-oxo-1,6-dihydropyrimidin-1-ium chloride is obtained.

### Example 73

### Production of 4(3H)-pyrimidinone

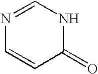

A mixture of 103 g (0.804 mol) 6-mercapto-4(1H)-pyrimidinone (JP 50053381, Chem. Abstr. CAN 84:17404) and 141.5 g (1.2 Mol) Raney nickel in 1.21 ethanol is heated for 8 hours under reflux. The solution is filtered hot, the residue is washed with ethanol, and the filtrate is concentrated under reduced pressure. 67.2 g (87% of theoretical yield) 4(3H)-pyrimidinone is obtained.
*) The logP values were determined in accordance with EEC directive 79/831 Annex V. A8 through HPLC (gradient method, acetonitrile/0.1% aqueous phosphoric acid).

### Example 74

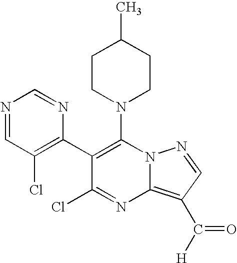

A mixture made of 5 mmol 5,7-dichloro-6-(5-chloro-pyrimidin-4-yl)-3-formyl-pyrazolo[1,5-a]-pyrimidine, 5 mmol 4-methylpiperidine and 5 mmol potassium carbonate in 30 ml acetonitrile wird is stirred 15 hours at room temperature. The reaction mixture is then poured into 120 ml water. The mixture is extracted three times using acetic ethyl ester, the combined organic phases are dried over sodium sulphate and concentrated under reduced pressure. The remaining residue is chromatographed using cyclohexane/acetic ethylester=3:1 on silica gel. In this way, 1.15 mmol of 5-chloro-6-(5-chloro-pyrimidin-4-yl)-3-formyl-7-(4-methyl-piperidin-1-yl)-pyrazolo[1,5-a]-pyrimidine is obtained.

HPLC: log P=3.04

### Example 75

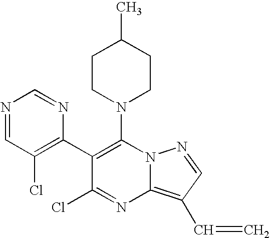

1.3 mmol 5-chloro-6-(5-chloro-pyrimidin-4-yl)-3-formyl-7-(4-methyl-piperidin-1-yl)-pyrazolo[1,5-a]pyrimidine is added to a solution of 1.4 mmol methyltriphenyl phosphonium bromide and 1.4 mmol n-butyl lithium in 58 ml tetrahydrofuran at −70° C. while stirring. The mixture is stirred a further 15 hours at room temperature, the solvent is then distilled off under reduced pressure and the residue is admixed with water.

The resulting mixture is extracted three times using acetic ethylester. The combined organic phases are dried over sodium sulphate and then concentrated under reduced pressure. The remaining residue is chromatographed using cyclohexane/acetic ethylester=7:3 on silica gel. In this way, 0.2 mmol of 5-chloro-6-(5-chloro-pyrimidin-4-yl)-3-ethenyl-7-(4-methyl-piperidin-4-yl)-pyrazolo[1,5-a]pyrimidine is obtained.

HPLC: log P=4.70

### Example 76

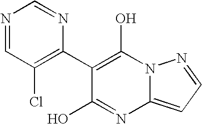

100 mmol 3-amino pyrazole and 100 mmol 2-(5-chloro-pyrimidin-4-yl)-malonic dimethylester are added to 27 ml tri-n-butyl-amine at room temperature while stirring. After admixing, the reaction mixture is heated 3 hours to 185° C. while stirring. The methanol produced during the reaction is continuously distilled off. The mixture is then cooled to room temperature, decanted off from tri-n-butyl amine, the residue is stirred with a mixture made of isopropanol and methyl tert.-butyl ether and decanted again. Still remaining residues of solvent are removed under reduced pressure. The 5,7-dihydroxy-6-(5-chloro-pyrimidin-4-yl)-pyrazolo[1,5-a]pyrimidine obtained is used for further reaction without additional purification.

### Example 77

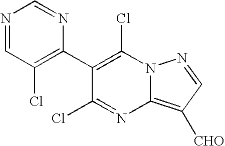

A mixture of 56 mmol 5,7-dihydroxy-6-(5-chloro-pyrimidin-4-yl)-pyrazolo[1,5-a]pyrimidine and 560 mmol phosphorus oxychloride is stirred 30 minutes at 30° C., then cooled to 0° C. and then admixed in drops with 85 mmol dimethyl formamide while stirring. After admixing, the reaction mixture is first stirred 12 hours at room temperature and then heated 6 hours under reflux. The reaction mixture is then admixed with 56 mmol phosphorus pentachloride and heated a further 12 hours under reflux. After cooling to room temperature, the reaction mixture is concentrated under reduced pressure and then poured onto ice water. The resulting mixture is extracted three times using acetic ethyl ester. The combined organic phases are dried over sodium sulphate and then concentrated under reduced pressure. The 3-formyl-5,7-dichloro-6-(5-chloro-pyrimidin-4yl)-pyrazolo[1,5-a]pyrimidine obtained is used for further synthesis without additional purification.

### Example A

Venturia Test (Apple)/Protective

Solvent: 24.5 parts by weight acetone
   24.5 parts by weight dimethylacetamide
Emulsifier: 1 part by weight alkyl-aryl polyglycolether

To produce an effective active ingredient preparation, 1 part by weight active ingredient is mixed with the specified quantities of solvent and emulsifier and the concentrate is diluted using water to the desired concentration.

To test for protective activity, young plants are sprayed with the active ingredient preparation in the specified applied quantity. After drying of the spray coating, the plants are inoculated with an aqueous conidia suspension of the apple scab pathogen Venturia inaequalis and then remain 1 day in an incubation chamber at approximately 20° C. and a relative ambient humidity of 100%.

The plants are then placed in a greenhouse at approximately 21° C. and a relative ambient humidity of approximately 90%.

The analysis is performed 10 days after the inoculation. In this case, 0% means an activity which corresponds to that of the control, while an activity of 100% means that no infection is observed.

In this test, the materials according to the present invention listed in Examples 1, 2, 3, 4 and 5 display an activity of over 90% at an applied quantity of 100 g/ha.

### Example B

Botrytis Test (Beans)/Protective

Solvent: 24.5 parts by weight acetone
   24.5 parts by weight dimethylacetamide
Emulsifier: 1 part by weight alkyl-aryl polyglycolether

To produce an effective active ingredient preparation, 1 part by weight active ingredient is mixed with the specified quantities of solvent and emulsifier and the concentrate is diluted using water to the desired concentration.

To test for protective activity, young plants are sprayed with the active ingredient preparation in the specified applied quantity. After drying of the spray coating, 2 small agar pieces covered with Botrytis cinerea are then laid on each leaf. The inoculated plants are then placed in a darkened chamber at approximately 20° C. and a relative ambient humidity of 100%.

The size of the infection spots on the leaves are analyzed 2 days after the inoculation. In this case, 0% means an activity which corresponds to that of the control, while an activity of 100% means that no infection is observed.

In this test, the materials according to the present invention listed in Examples 2, 3 and 5 display an activity of over 85% at an applied quantity of 500 g/ha.

### Example C

Puccinia Test (Wheat)/Protective

Solvent: 50 parts by weight N,N-dimethyl formamide
Emulsifier: 1 part by weight alkyl aryl polyglycolether

To produce an effective active ingredient preparation, 1 part by weight active ingredient is mixed with the specified quantities of solvent and emulsifier and the concentrate is diluted using water to the desired concentration.

To test for protective activity, young plants are sprayed with the active ingredient preparation in the specified applied quantity. After drying of the spray coating, the plants are sprayed with a conidia suspension of Puccinia recondita. The plants remain 48 hours at 20° C. and 100% relative ambient humidity in an incubation chamber.

The plants are placed in a greenhouse at a temperature of approximately 20° C. and a relative ambient humidity of approximately 80% in order to encourage the development of rust pustules.

The analysis is performed 10 days after the inoculation. In this case, 0% means an activity which corresponds to that of the control, while an activity of 100% means that no infection is observed.

In this test, the materials according to the present invention listed in Examples 2 and 39 display an activity of over 85% at an applied quantity of 500 g/ha.

### Example D

Podosphaera Test (Apple)/Protective

Solvent: 24.5 parts by weight acetone
   24.5 parts by weight dimethylacetamide
Emulsifier: 1 part by weight alkyl-aryl polyglycolether

To produce an effective active ingredient preparation, 1 part by weight active ingredient is mixed with the specified quantities of solvent and emulsifier and the concentrate is diluted using water to the desired concentration.

To test for protective activity, young plants are sprayed with the active ingredient preparation in the specified applied quantity. After drying of the spray coating, the plants are inoculated with an aqueous spore suspension of the apple powdery mildew pathogen Podosphaera leucotricha. The plants are then placed in a greenhouse at approximately 23° C. and a relative ambient humidity of approximately 70%.

The analysis is performed 10 days after the inoculation. In this case, 0% means an activity which corresponds to that of the control, while an activity of 100% means that no infection is observed.

In this test, the materials according to the present invention listed in Examples 3 and 5 display an activity of over 90% at an applied quantity of 100 g/ha.

### Example E

In Vitro Test to Determine the ED₅₀ on Micro-Organisms

Solvent: methanol
Emulsifier: alkylaryl polyglycol ether

To produce an expedient active ingredient preparation, 2 mg active ingredient is mixed with 100 μl methanol and the concentrate thus produced is diluted using a mixture of 1000 ml methanol and 6 g of the above-mentioned emulsifier to the particular desired concentration.

10 μl is pipetted into each of the cavities of microtitration plates. After the solvent has evaporated, 200 μl of a potato dextrose medium, which had previously been admixed with the particular desired concentration of spores and/or mycelia of the micro-organisms to be tested, is added to each of the cavities. The resulting concentrations of active ingredient in the cavities are

| | |
|---|---|
| 0.1 | ppm |
| 1 | ppm |
| 10 | ppm |
| and 100 | ppm, respectively. |

The resulting concentration of emulsifier is 300 ppm in each case.

For incubation, the microtitration plates are subsequently moved 3 to 5 days on a shaker at a temperature of 22° C. until a sufficient growth of the particular micro-organism may be determined in the untreated control.

The analysis is performed photometrically at a wavelength of 620 nm. The active ingredient dose which results in a 50% inhibition of the fungi growth (ED₅₀) in relation to the untreated control is calculated from the measured data for the different concentrations.

In this test, the ED₅₀ value of the compound according to the present invention listed in Example 1 for Botrytis cinerea is at an active ingredient dose which is lower than 10 ppm.

1. A compound of the formula 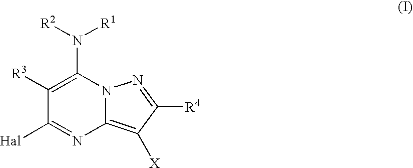
in which
R¹ represents optionally substituted alkyl, optionally substituted alkenyl, optionally substituted alkynyl, optionally substituted cycloalkyl, or optionally substituted heterocyclyl,
R² represents hydrogen or alkyl, or
R¹ and R² together with the nitrogen atom to which they are bound, represent an optionally substituted heterocyclic ring,
R³ represents optionally substituted heterocyclyl,
R⁴ represents hydrogen or alkyl,
Hal represents halogen and
X represents halogen, cyano, nitro, alkyl, optionally substituted alkenyl, optionally substituted alkynyl, hydroxyalkyl, alkoxyalkyl, halogenalkyl, cycloalkyl, formyl, thiocarbamoyl, alkoxycarbonyl, alkylcarbonyl, hydroxyiminoalkyl, alkoximinoalkyl, alkylthio, alkylsulphinyl, alkylsulphonyl or alkylaminocarbonyl.

2. The compound of the formula (I) according to claim 1, in which
R¹ represents alkyl having 1 to 6 carbon atoms, which may be substituted one to five times, identically or differently, by halogen, cyano, hydroxy, alkoxy having 1 to 4 carbon atoms and/or cycloalkyl having 3 to 6 carbon atoms, or
R¹ represents alkenyl having 2 to 6 carbon atoms, which may be substituted one to three times, identically or differently by halogen, cyano, hydroxy, alkoxy having 1 to 4 carbon atoms and/or cycloalkyl having 3 to 6 carbon atoms, or
R¹ represents alkynyl having 2 to 6 carbon atoms, which may be substituted one to three times, identically or differently by halogen, cyano, alkoxy having 1 to 4 carbon atoms and/or cycloalkyl having 3 to 6 carbon atoms, or
R¹ represents cycloalkyl having 3 to 6 carbon atoms, which may be substituted one to three times, identically or differently by halogen and/or alkyl having 1 to 4 carbon atoms, or
R¹ represents saturated or unsaturated heterocyclyl having 5 or 6 ring members and 1 to 3 heteroatoms, selected from the group consisting of nitrogen, oxygen, and sulphur, the heterocyclyl able to be substituted once or twice by halogen, alkyl having 1 to 4 carbon atoms, cyano, nitro and/or cycloalkyl having 3 to 6 carbon atoms,
R² represents hydrogen or alkyl having 1 to 4 carbon atoms, or
R¹ and R² together with the nitrogen atom to which they are bound, represent a saturated or unsaturated heterocyclic ring having 3 to 6 ring elements, the heterocyclic compound able to contain a further nitrogen, oxygen, or sulphur atom as a ring element and the heterocyclic compound able to be substituted up to three times by fluoride, chloride, bromide, nitro, alkyl having 1 to 4 carbon atoms and/or halogenalkyl having 1 to 4 carbon atoms and 1 to 9 fluorine and/or chlorine atoms,
R³ represents saturated or unsaturated heterocyclyl having 5 or 6 ring members and 1 to 4 heteroatoms, selected from the group consisting of oxygen, nitrogen and sulphur, the heterocyclyl being able to be substituted one to four times, identically or differently by fluoride, chloride, bromide, cyano, nitro, alkyl, alkoxy, hydroximinoalkyl or alkoximinoalkyl each having 1 to 3 carbon atoms in each alkyl part, halogenalkyl or halogenalkoxy each having 1 to 3 carbon atoms and 1 to 7 halogen atoms,
R⁴ represents hydrogen or alkyl having 1 to 4 carbon atoms
Hal represents fluoride, chloride, or bromide and
X represents cyano, fluoride, chloride, bromide, iodide, nitro, formyl, halogenalkyl having 1 to 6 carbon atoms and 1 to 9 fluoride, chloride and/or bromide atoms, alkyl having 1 to 4 carbon atoms, alkenyl having 2 to 6 carbon atoms, alkenyl, substituted by carboxyl, methoxycarbonyl, or ethoxycarbonyl, having 2 to 5 carbon atoms in the alkenyl part, alkynyl having 2 to 6 carbon atoms, alkenyl, substituted by carboxyl, methoxycarbonyl, or ethoxycarbonyl, having 2 to 5 carbon atoms in the alkynyl part, hydroxyalkyl having 1 to 4 carbon atoms, alkoxyalkyl having 1 to 4 carbon atoms in the alkoxy part and 1 to 4 carbon atoms in the alkyl part, cycloalkyl having 3 to 6 carbon atoms, thiocarbamoyl, alkoxycarbonyl having 1 to 4 carbon atoms in the alkoxy part, alkylcarbonyl having 1 to 4 carbon atoms in the alkyl part, hydroximinoalkyl having 1 to 4 carbon atoms in the alkyl part, alkoximinoalkyl having 1 to 4 carbon atoms in the alkoxy part and 1 to 4 carbon atoms in the alkyl part, alkylthio having 1 to 4 carbon atoms, alkylsulphinyl having 1 to 4 carbon atoms, alkylsulphonyl having 1 to 4 carbon atoms or alkylaminocarbonyl having 1 to 4 carbon atoms in the alkyl part.

3. A compound of the formula (I) according to claim 1 or 2, in which
R¹ represents a residue of the formula 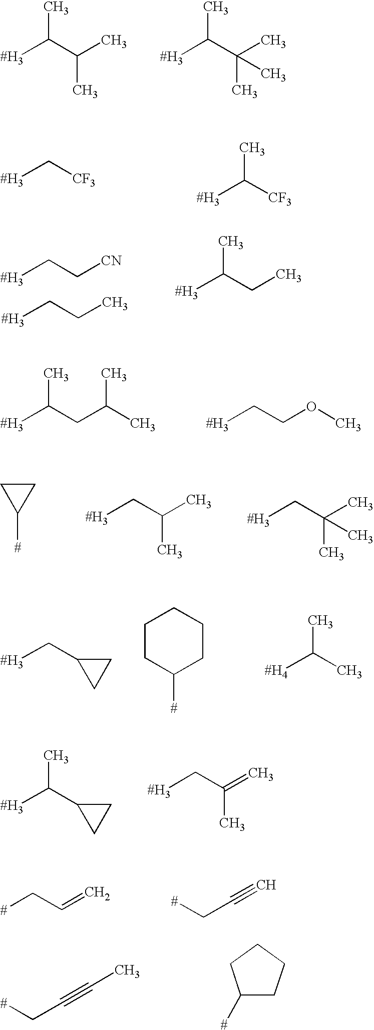
# marking the linkage point,
R² represents hydrogen, methyl, ethyl or propyl, or
R¹ and R² together with the nitrogen atom to which they are bound, represent pyrrolidinyl, piperidinyl, morpholinyl, thiomorpholinyl, piperazinyl, 3,6-dihydro-1(2H)-piperidinyl or tetrahydro-1(2H)-pyridazinyl, these residues being able to be substituted by 1 to 3 fluoride atoms, 1 to 3 methyl groups and/or trifluoromethyl,
or
R¹ and R² together with the nitrogen atom to which they are bound, represent a residue of the formula 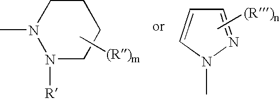
in which
R′ represents hydrogen or methyl,
R″ represents methyl, ethyl, fluorine, chlorine or trifluoromethyl,
m represents the numbers 0, 1, 2 or 3, R″ representing identical or different residues if m represents 2 or 3,
R′″ represents methyl, ethyl, fluorine, chlorine or trifluoromethyl and
n represents the numbers 0, 1, 2 or 3, R′″ representing identical or different residues if n represents 2 or 3,
R³ represents pyridyl, which is linked in the second or fourth position and may be substituted one to four times, identically or differently, by fluoride, chloride, bromide, cyano, nitro, methyl, ethyl, methoxy, methylthio, hydroximinomethyl, hydroximinoethyl, methoximinomethyl, methoximinoethyl and/or trifluoromethyl, or
R³ represents pyrimidyl, which is linked in the second or fourth position and may be substituted one to three times, identically or differently, by fluoride, chloride, bromide, cyano, nitro, methyl, ethyl, methoxy, methylthio, hydroximinomethyl, hydroximinoethyl, methoximinomethyl, methoximinoethyl and/or trifluoromethyl, or
R³ represents thienyl, which is linked in the second or third position and may be substituted one to three times, identically or differently, by fluoride, chloride, bromide, cyano, nitro, methyl, ethyl, methoxy, methylthio, hydroximinomethyl, hydroximinoethyl, methoximinomethyl, methoximinoethyl and/or trifluoromethyl, or
R³ represents thiazolyl, which is linked in the second, fourth, or fifth position and may be substituted once or twice, identically or differently, by fluoride, chloride, bromide, cyano, nitro, methyl, ethyl, methoxy, methylthio, hydroximinomethyl, hydroximinoethyl, methoximinomethyl, methoximinoethyl and/or trifluoromethyl,
R⁴ represents hydrogen, methyl, ethyl, propyl or isopropyl
Hal represents fluoride or chloride and
X represents cyano, fluoride, chloride, bromide, iodide, nitro, formyl, trifluoromethyl, difluoromethyl, methyl, ethyl, cyclopropyl, thiocarbamoyl, methoxycarbonyl, methylcarbonyl, ethylcarbonyl, hydroximinomethyl, methoximinomethyl, methylthio, methylsulphinyl, methylsulphonyl, methylaminocarbonyl, ethenyl, propenyl, hydroxymethyl, hydroxyeth-1-yl, methoxymethyl, thoxymethyl or 1-methoxy-ethyl, or
X represents a residue of the formula

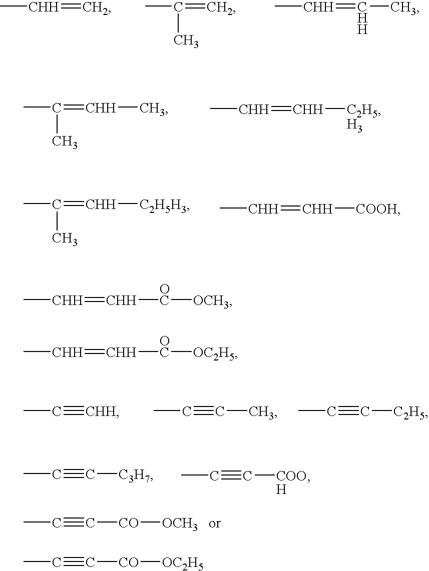

4. A method for producing compounds of the formula (I) according to claim 1, characterized in that
a) one or more compounds of the formula 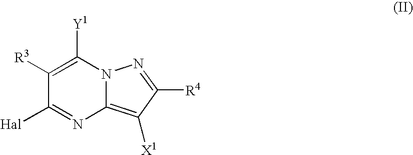
in which
R³, R⁴, and Hal have the meanings specified above,
X¹ represents halogen, cyano, nitro, alkyl, halogenalkyl, cycloalkyl, formyl, thiocarbamoyl, alkoxycarbonyl, alkylcarbonyl, alkylthio, alkylsulphinyl, alkylsulphonyl or alkylaminocarbonyl and
Y¹ represents halogen,
are reacted with one or more compounds of the formula 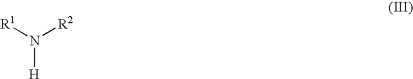
in which
R¹ and R² have the meanings specified above, optionally in the presence of a diluent, optionally in the presence of a catalyst, and optionally in the presence of an acid acceptor,
or
b) one or more compounds of the formula 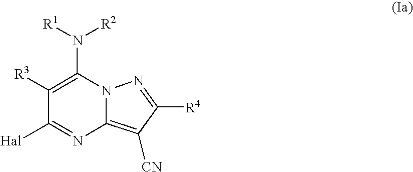
in which
R¹, R², R³, R⁴, and Hal have the meanings specified above,
either
a) are reacted with diisobutyl aluminum hydride in the presence of aqueous ammonium chloride solution and in the presence of an organic diluent,
or
b) are reacted with one or more compounds of the formula
R⁵—Mg—X² (IV)
in which
R⁵ represents alkyl
X² represents chloride or bromide,
in the presence of a diluent and optionally in the presence of a catalyst,
or
c) one or more compounds of the formula 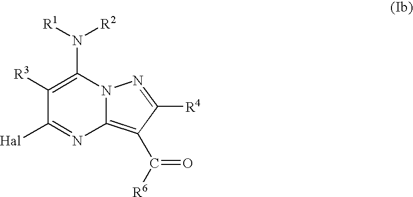
in which
R¹, R², R³, R⁴, and Hal have the meanings specified above and
R⁶ represents hydrogen or alkyl,
either
a) are reacted with one or more compounds of the formula
H₂N—OR⁷ (V)
in which
R⁷ represents hydrogen or alkyl,
in the presence of a diluent and optionally in the presence of a catalyst, the compounds of the formula (V) also being able to be used in the form of their acid addition salts,
or
b) are reacted with one or more compounds of the formula 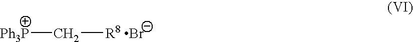
in which
Ph represents phenyl and
R⁸ represents hydrogen or optionally substituted alkyl,
in the presence of a base and in the presence of a diluent,
or
c) are reacted with diisobutyl aluminum hydride in the presence of aqueous ammonium chloride solution and in the presence of an organic diluent, or are reacted with sodium borohydride in the presence of a diluent, and optionally the resulting compounds of the formula 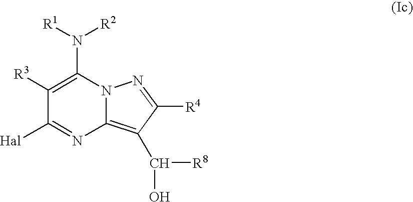
in which
R¹, R², R³, R⁴, R⁸, and Hal have the meanings specified above,
are reacted with one or more compounds of the formula
R⁹—X³ (VII)
in which
R⁹ represents alkyl
X³ represents chloride, bromide, iodide or the residue R⁹⁰—SO₂—O—, optionally in the presence of a base and in the presence of a diluent,
or
d) compounds of the formula 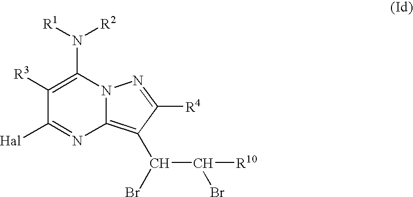
in which
R¹, R², R³, R⁴ and Hal have the meanings specified above,
R¹⁰ represents hydrogen or optionally substituted alkyl,
are reacted with strong bases in the presence of a diluent,
or
e) compounds of the formula 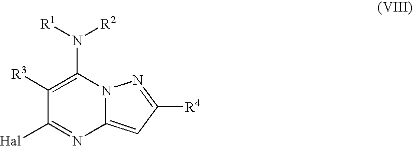
in which
R¹, R², R³, R⁴ and Hal have the meanings specified above,
are reacted with one or more compounds of the formula 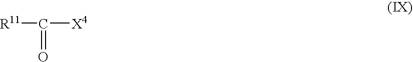
in which
R¹¹ represents alkyl and
X⁴ represents chloride or a residue of the formula 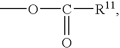
in the presence of a catalyst and in the presence of a diluent.

5. A composition comprising at least one compound of the formula (I) according to claim 1, in addition to extenders and/or surfactants.

6. (canceled)

7. A method for combating undesired, micro-organisms, comprising applying one or more compounds of the formula (I) according to claim 1 to the undesired micro-organisms and/or their living space.

8. A method for producing the composition of claim 5, comprising contacting said compounds of the formula (I) with said extenders and/or surfactants.

9. A compound of the formula 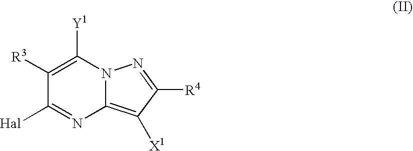
in which
R³ represents optionally substituted heterocyclyl,
R⁴ represents hydrogen or alkyl,
Hal represents halogen,
X¹ represents halogen, cyano, nitro, alkyl, halogenalkyl, cycloalkyl, formyl, thiocarbamoyl, alkoxycarbonyl, alkylcarbonyl, alkylthio, alkylsulphinyl, alkylsulphonyl or alkylaminocarbonyl and
Y¹ represents halogen.

10. A method for producing compounds of the formula (II) according to claim 9, characterized in that
f) one or more compounds of the formula 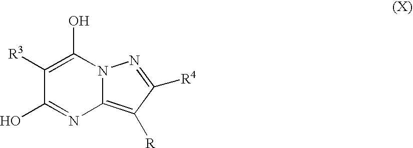
in which
R³ and R⁴ have the meanings specified in claim 9, and
R represents halogen, cyano, nitro, alkyl, halogenalkyl, cycloalkyl, thio carbamoyl, alkoxycarbonyl, alkylthio, alkylsulphinyl, alkylsulphonyl or alkylaminocarbonyl,
are reacted with halogenation agents, optionally in the presence of a diluent,
or
g) one ore more compounds of the formula 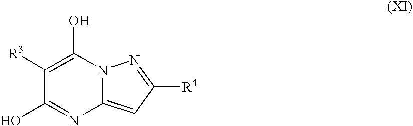
in which
R³ and R⁴ have the meanings specified in claim 9,
are reacted with phosphorus oxychloride in the presence of dimethyl formamide and optionally reacted further while adding phosphorus pentachloride.

11. A compound of the formula 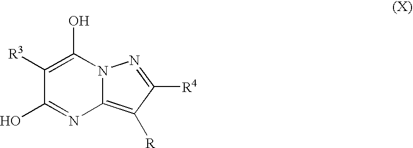
in which
R³ represents optionally substituted heterocyclyl,
R⁴ represents hydrogen or alkyl steht and
R represents halogen, cyano, nitro, alkyl, halogenalkyl, cycloalkyl, thio carbamoyl, alkoxycarbonyl, alkylthio, alkylsulphinyl, alkylsulphonyl or alkylaminocarbonyl.

12. A method for producing compounds of the formula (X) according to claim 11, characterized in that
(h) one or more compounds of the formula 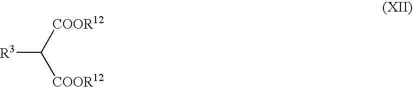
in which
R³ has the meaning specified in claim 11 and
R¹² represents alkyl having 1 to 4 carbon atoms,
are reacted with one ore more compounds of the formula 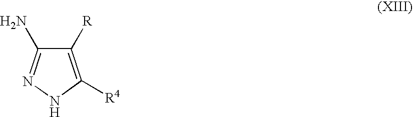
in which
R⁴ and R have the meanings specified in claim 11,
optionally in the presence of a diluent and optionally in the presence of an acid binder.

13. A compound of the formula 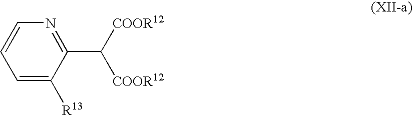
in which
R¹² represents alkyl having 1 to 4 carbon atoms and
R¹³ represents halogen or halogenalkyl.

14. A method for producing compounds of the formula (XII-a) according to claim 13, characterized in that
(i) one or more compounds of the formula 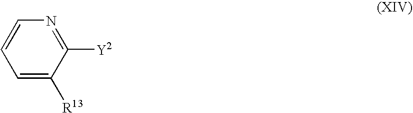
in which
R¹³ has the meaning specified in claim 13 and
Y² represents halogen,
are reacted with one or more compounds of the formula 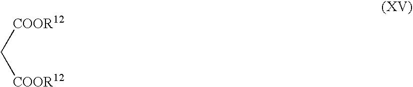
in which
R¹² has the meaning specified in claim 13,
optionally in the presence of a diluent, optionally in the presence of a copper salt and optionally in the presence of an acid acceptor.

15. A compound of the formula 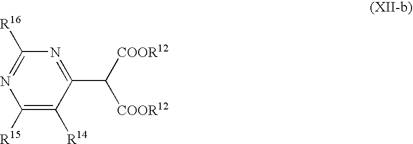
in which
R¹² represents alkyl having 1 to 4 carbon atoms,
R¹⁴ represents halogen or halogen alkyl, and
R¹⁵ and R¹⁶ independently of one another, represent hydrogen, fluoride, chloride, bromide, methyl, ethyl or methoxy.

16. A method for producing compounds of the formula (XII-b) according to claim 15, characterized in that
(j) one or more compounds of the formula 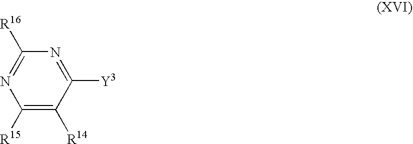
in which
R¹⁴, R¹⁵ and R¹⁶ have the meanings specified in claim 15 and
Y³ represents halogen,
are reacted with one or more compounds of the formula 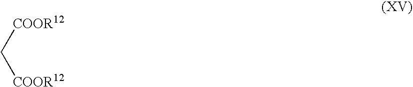
in which
R¹² has the meaning specified in claim 15,
optionally in the presence of a diluent, optionally in the presence of a copper salt and optionally in the presence of an acid acceptor.
